(19) United States
(12) Patent Application Publication
(10) Pub. No.: US 2025/0352405 A1
(43) Pub. Date: Nov. 20, 2025

(54) TAMPON INCLUDING SECONDARY ABSORBENT

(71) Applicant: THE PROCTER & GAMBLE COMPANY, Cincinnati, OH (US)

(72) Inventors: Khalid QURESHI, Mason, OH (US); Holly Balasubramanian RAUCKHORST, Fort Thomas, KY (US); Gerald Westley BROWN, III, South Portland, ME (US); Ryo MINOGUCHI, Cincinnati, OH (US); Paul Thomas WEISMAN, Cincinnati, OH (US); Eric Mitchell SCHICKLI, Cincinnati, OH (US)

(21) Appl. No.: 19/206,510

(22) Filed: May 13, 2025

Related U.S. Application Data
(60) Provisional application No. 63/647,793, filed on May 15, 2024.
(60) Provisional application No. 63/754,662, filed on Feb. 6, 2025.

(51) Int. Cl. A61F 13/20; A61F 13/15; A61F 13/34; A61L 15/28; A61L 15/40

(52) U.S. Cl. CPC A61F 13/2071; A61F 13/2051; A61F 13/34; A61L 15/28; A61L 15/40; A61F 2013/15365

(57) ABSTRACT

A tampon having an insertion end and a withdrawal end. The tampon includes a primary absorbent, disposed toward the insertion end, a secondary absorbent, and a withdrawal member. The secondary absorbent may have a mean length from the primary absorbent and at least three segments each having a mean segment mass. The mean segment mass may be selected such that the secondary absorbent tapers as the secondary absorbent material extends from the primary absorbent. The primary absorbent including at least one of rayon fibers and cotton fibers and having improved fluid handling due to dynamic expansion of the tampon at a relatively reduced dry tampon mass.

### CROSS REFERENCE TO RELATED APPLICATIONS

This application claims the benefit, under 35 U.S.C. § 119(e), to U.S. Provisional Application Nos. 63/647,793 filed May 15, 2024, and 63/754,662 filed Feb. 6, 2025, the entire disclosures of which are fully incorporated by reference herein.

### FIELD OF THE INVENTION

The present disclosure relates to disposable absorbent articles, more specifically disposable feminine hygiene absorbent articles, including absorbent tampons having a secondary absorbent.

### BACKGROUND OF THE INVENTION

A variety of designs for absorbent tampons have been manufactured for a number of years and used by women to capture and absorb menstrual fluid internally, in conjunction with, or as an alternative to, externally worn feminine hygiene pads. Many women prefer to use tampons as an alternative to feminine hygiene pads at least some of the time during menstruation, because, among other reasons, tampons are used internally and are thereby discreet, creating none of the bulk under clothing that is associated with many types of feminine hygiene pads.

Particularly when a feminine hygiene pad is not used in conjunction therewith, it is important that the tampon capture and absorb most if not substantially all of the menstrual fluid that is discharged during the tampon's usage duration (to the extent of its absorption capacity), to help avoid a leakage of fluid that may soil underwear, outer clothing, bedclothes, etc. The prior art has recognized various ways in which tampons might fail to perform effectively. One such way is sometimes referred to as “bypass” failure or bypass leakage. Bypass failure occurs when the menstrual fluid travels along the length of the vaginal cavity without contacting the tampon, or the tampon has available absorption capacity but fails to capture and absorb the fluid.

A variety of approaches to tampon design have sought to mitigate such leakage. One approach that has proven effective has been to include a secondary absorbent as part of the withdrawal member. The secondary absorbent is a material/structure selected and configured to extend downward (or trail, to the rear of) the main pledget or primary absorbent, along with the withdrawal member, thereby extending further down the vaginal cavity toward the vaginal opening than the pledget, following insertion. Appropriately configured, the secondary absorbent can engage menstrual fluid flowing along the vaginal cavity past the pledget, capture it, and wick it back to the pledget. Current approaches to manufacture of such tampons, however, have shown to be inefficient, and current selections of configurations and materials have shown to be less effective than may be fully realized.

Accordingly, there remains an opportunity for improvement in the construction of tampons with secondary absorbents.

### SUMMARY OF THE INVENTION

In some embodiments, an absorbent tampon having an insertion end and a withdrawal end comprises a primary absorbent, disposed toward the insertion end, wherein the primary absorbent comprises cotton fibers and rayon fibers, and wherein the primary absorbent has an expansion width of greater than 20 mm; a secondary absorbent; and a withdrawal member. The secondary absorbent may exhibit a mean length from the primary absorbent of from about 20 mm to about 40 mm, and the secondary absorbent may exhibit a Mean Segment Mass at a length of 20-30 mm from the primary absorbent of greater than about 9.0 mg. The mean length from the primary absorbent and mean segment mass are each measured according to the Off-Pad Secondary Absorbent Profile Method as described herein.

In some embodiments, an absorbent tampon having an insertion end and a withdrawal end comprises a primary absorbent, disposed toward said insertion end, wherein the primary absorbent comprises less than 50% by weight cotton fibers and greater than 50% by weight rayon fibers, and wherein the primary absorbent has an expansion width of greater than 20 mm; a secondary absorbent; and a withdrawal member. The secondary absorbent may exhibit a mean length from the primary absorbent of from about 20 mm to about 40 mm, and the secondary absorbent may exhibit a mean segment mass at a length of 20-30 mm from the primary absorbent of from about 5.0 mg to about 30 mg. Additionally, the secondary absorbent may exhibit a standard deviation of the mean segment mass at the length of 20-30 mm from the primary absorbent of less than about 7 mg. The mean length from the primary absorbent, mean segment mass, and the standard deviation of the mean segment mass are measured according to the Off-Pad Secondary Absorbent Profile method as described herein.

In some embodiments, an absorbent tampon having an insertion end and a withdrawal end comprises a primary absorbent, disposed toward the insertion end, wherein the primary absorbent comprises less than 80% by weight cotton fibers and greater than 20% by weight rayon fibers, and wherein the primary absorbent has an expansion width of greater than 20 mm; a secondary absorbent; and a withdrawal member. The secondary absorbent may exhibit a mean length from the primary absorbent of from about 20 mm to about 40 mm, and the secondary absorbent may comprise a first segment having a length from the primary absorbent of 0 to 10 mm, a second segment having a length from the primary absorbent of from 10 mm to 20 mm, and a third segment having a length from the primary absorbent of from 20 mm to 30 mm. The difference between a mean segment mass of the third segment and the mean segment mass of the fourth segment may be at least 8 mg. The mean length from the primary absorbent and mean segment mass are measured according to the Off-Pad Secondary Absorbent Profile method as described herein.

### BRIEF DESCRIPTION OF THE DRAWINGS

FIG. 1 is a longitudinal side view of an example of a tampon with a primary absorbent shaped and compressed into a self-sustaining form.

FIG. 2 is a longitudinal front side view of an example of a tampon before the primary absorbent has been shaped and compressed into a self-sustaining form.

FIG. 3A is a lateral cross section of the tampon shown in FIG. 2, taken through line 3A-3A in FIG. 2.

FIG. 3B is an exploded longitudinal cross section of components of the tampon shown in FIG. 2, taken through line 3B-3B in FIG. 2, and shown with the components separated, i.e., not joined together by stitching or other joining mechanism.

FIG. 4A is a longitudinal cross section of portions of a wicking member and withdrawal cord of the tampon shown in FIG. 2, taken through line 4-4 in FIG. 2.

FIG. 4B is an enlarged view of portion of lockstitching shown within circle 4B in FIG. 4A.

FIG. 5A is a longitudinal side view of an example of a tampon including a primary absorbent, a secondary absorbent, and a withdrawal member, and the secondary absorbent is divided into segments.

FIG. 5B is a graphical representation of the Off-Pad Secondary Absorbent Profile for various absorbent tampons.

FIG. 6A is a lateral cross section of an example of a primary absorbent prior to attachment of a withdrawal member.

FIG. 6B is a lateral cross section of an example of a tampon including the primary absorbent shown in FIG. 6A, following attachment of a withdrawal member via stitching.

FIG. 6C is a lateral cross section of the tampon shown in FIG. 6B, shown at a time proximate commencement of folding as a step to formation of the primary absorbent into a self-sustaining form.

FIG. 6D is a lateral cross section of the tampon shown in FIG. 6B, shown at a time during folding as a step to formation of the primary absorbent into a self-sustaining form.

FIG. 6E is a lateral cross section of the tampon shown in FIG. 6B, shown at a time following folding and during radial compression as a step to formation of the primary absorbent into a self-sustaining form.

FIG. 7 is a schematic depiction of a configuration of equipment used in the Tampon Pusher Drag Force method herein.

FIGS. 8A and 8B are schematic depictions of a configuration of equipment used in the Wicking Measurement method herein.

FIG. 9 is a schematic diagram showing an apparatus for measuring the dynamic expansion of tampons.

FIG. 10 is a schematic diagram of the apparatus of FIG. 9 with some of the features of FIG. 9 removed for case of visualization.

### DETAILED DESCRIPTION

As used herein the term “tampon” refers to any type of absorbent structure which is inserted into the vaginal canal for the absorption of fluid therefrom. Typically, a tampon includes a primary absorbent, also referred to herein as a pledget, including a quantity of absorbent material, often absorbent fibrous material. The primary absorbent structure has been bunched, folded and/or compressed in one or more radial directions, the longitudinal direction, or both, via application of pressure, heat and moisture control, in order to provide a formed tampon having a size, shape and stability of form to facilitate insertion into the vagina. A tampon which has been so formed is referred to herein has a “self-sustaining” form. The degree of compression, heat and moisture control applied to the primary absorbent is sufficient such that in the subsequent absence of the external forces and absence of substantial contact with moisture, the primary absorbent will tend to retain its general formed shape and size.

It will be understood by persons of ordinary skill in the art that this self-sustaining form typically does not persist following insertion of the tampon. Once the tampon is inserted and begins to contact and absorb fluid, the primary absorbent will swell with absorbed fluid, expand, and lose its self-sustaining form.

As used herein the terms “primary absorbent” or “pledget” are intended to be interchangeable and refer to a structure including absorbent material configured to perform the primary function of the tampon, absorption of menstrual fluid. A tampon pledget is sometimes referred to as a tampon blank, or a softwind, and the term “primary absorbent” and “pledget” are intended to include structures designated by such terms as well.

As used herein the terms “vaginal cavity,” “within the vagina” and “vaginal interior,” are intended to be synonymous and refer to the internal genitalia of the human female in the pudendal region of the body. The term “vaginal cavity” as used herein is intended to refer to the space located between the introitus of the vagina (sometimes referred to as the sphincter of the vagina) and the cervix, and is not intended to include the interlabial space, including the floor of the vestibule. The external features of the female genitalia generally are not included within the term “vaginal cavity” as used herein.

With respect to a tampon, the “longitudinal” direction is the ordinary general direction of ejection from an applicator; and also corresponds with the ordinary general direction of insertion into and withdrawal from the vaginal cavity in normal use. For a completely manufactured, pre-use tampon that has a primary absorbent with a generally cylindrical or capsule-shaped self-sustaining form, the longitudinal axis of the form generally lies along the longitudinal direction. The “radial” or “lateral” direction is a direction perpendicular to the longitudinal direction. References to “length” herein refer to a dimension along the longitudinal direction; references to “width” herein refer to a dimension along the lateral direction.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

“Withdrawal member” refers to any section of string, yarn, cord, ribbon, strip material or other flexible/pliable elongate structure typically (although not necessarily) formed of fibrous material, attached to and/or extending from a tampon primary absorbent and trailing from its rearward end. A withdrawal member of sufficient length may be provided with a tampon for the purpose of providing a relatively thin and flexible trailing member of sufficient length to allow for a portion thereof to trail and remain outside of the introitus following full insertion of the tampon, which the user may easily grasp and pull to withdraw the tampon from her body following a desired duration of use.

The “syngyna” is an industry standard tampon absorbency capacity test. The apparatus and method for performing this tampon absorbency test are provided in 21 United States Code of Federal Regulation 801.430. In the United States and other regions, tampons carry standardized absorbency labels according to syngyna test absorbency ranges. Tampons absorbing less than 6 grams as “light absorbency,” tampons absorbing from 6 grams to 9 grams are labeled as “regular absorbency,” tampons absorbing 9 grams to 12 grams are labeled as “super absorbency,” tampons absorbing 12 grams to 15 grams are labeled as “super plus absorbency,” and tampons absorbing greater than 15 grams are labeled as “ultra absorbency.”

The present disclosure relates to an improved absorbent tampon provided with an improved secondary absorbent. An absorbent tampon comprising a secondary absorbent is disclosed by e.g., U.S. Pat. Nos. 6,258,075 and 6,599,279, which are herein incorporated by reference, and commercially available under the trade name of TAMPAX PEARL™. The secondary absorbent is located proximate the withdrawal end of the primary absorbent of the tampon and has been known to provide leakage protection by providing an additional absorbent material in the distal end of vaginal cavity to intercept the fluid that bypasses the primary absorbent before it leaks beyond the vaginal opening.

Ideally the secondary absorbent provides coverage of entire length of the vaginal cavity down to the vaginal opening across woman-to-woman anatomical variability of human anatomy. According to Luo, et al. “Quantitative Analyses of Variability in Normal Vaginal Shape and Dimension on MR Images.” International Urogynecology Journal, vol. 27, no. 7, 2016, pp. 1087-109, average vaginal length of about 80 healthy Caucasian women is about 8 cm (average of anterior and posterior wall lengths). If a primary absorbent of a tampon is about 4.5 cm long, then about 3.5 cm or more remains as vaginal length for the secondary absorbent to cover for ideal interception of the by-passed fluid. Importantly, achieving high performance levels of bypass leakage protection requires having the secondary absorbent in the vicinity or potentially within the vaginal opening itself, or even traversing outside the vaginal opening. However, the secondary absorbent has a separate total length limit to be met. The inventors have found that in the vaginal cavity, wearing discomfort may be experienced if the secondary absorbent is located at or traverse the vaginal opening because of nerves and/or sensitive tissue located proximate the vaginal opening. Ideally, the primary absorbent and the secondary absorbent are configured such that the primary absorbent region and the secondary absorbent region reside entirely within the vaginal space or cavity during use without extending outwardly therefrom.

The invention disclosed herein addresses this problem of how to achieve the highest performance combination of bypass leakage protection while being comfortable to wearer during use. The inventors have unexpectedly discovered the solution is to have select absorbent material levels and ranges associated with the secondary absorbent located at length position segments along its total length, and for the secondary material to be a select mean length and range. With such an inventive design combination, the overall tampon product may provide ideal coverage of the entire length of the vaginal cavity, especially the position at the vaginal opening and yet not have any wearing discomfort by, for example, irritating the nerves and/or sensitive tissues proximate the vaginal opening.

Further, the inventors have found to deliver tampon products which reliably exhibit this superior performance combination of bypass leakage protection with a comfortable wearing experience, the tampon product should have a relatively low variation in the select absorbent material levels associated with the secondary absorbent located at length position segments along its total length and the secondary material mean length. The invention encompasses tampon products exhibiting low standard deviations in these two characterizing parameters.

Additionally, in increasing the length of the secondary absorbent and changing the profile of the secondary absorbent to make it more uniform, the inventors needed to ensure that this would not adversely affect how the user interacted with the tampon product. In a tampon contained in an applicator that assists in insertion of the tampon into the vaginal cavity, the increased length of the secondary absorbent may be accompanied by an increase in the drag force of the secondary absorbent within the pusher of the applicator and disrupt overall smooth delivery of the tampon into the vaginal cavity. The inventors discovered the tampon products in an applicator with the inventive secondary absorbents as disclosed herein may provide acceptable insertion experiences by having a secondary absorbent design exhibiting a pusher peak drag force of less than 85 grams. Such tampon products contained within an applicator may deliver high performance insertion experience as the secondary absorbent exhibits a low enough drag force on the internal pusher wall to enable smooth comfortable expulsion of the tampon including the secondary absorbent out of the applicator barrel and pusher.

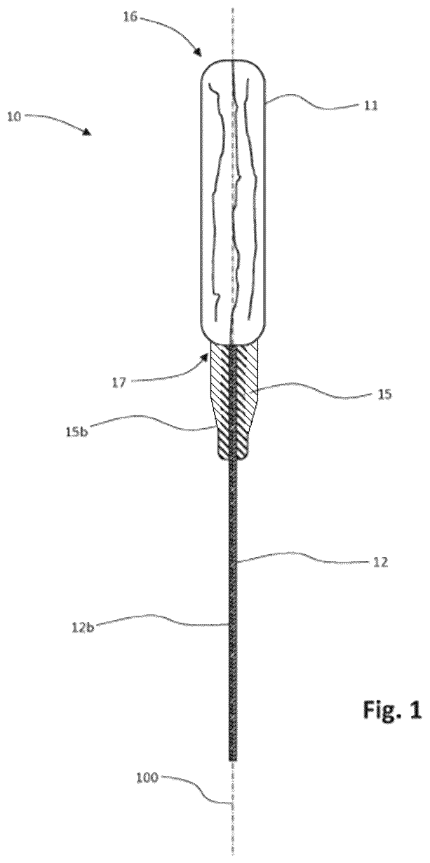

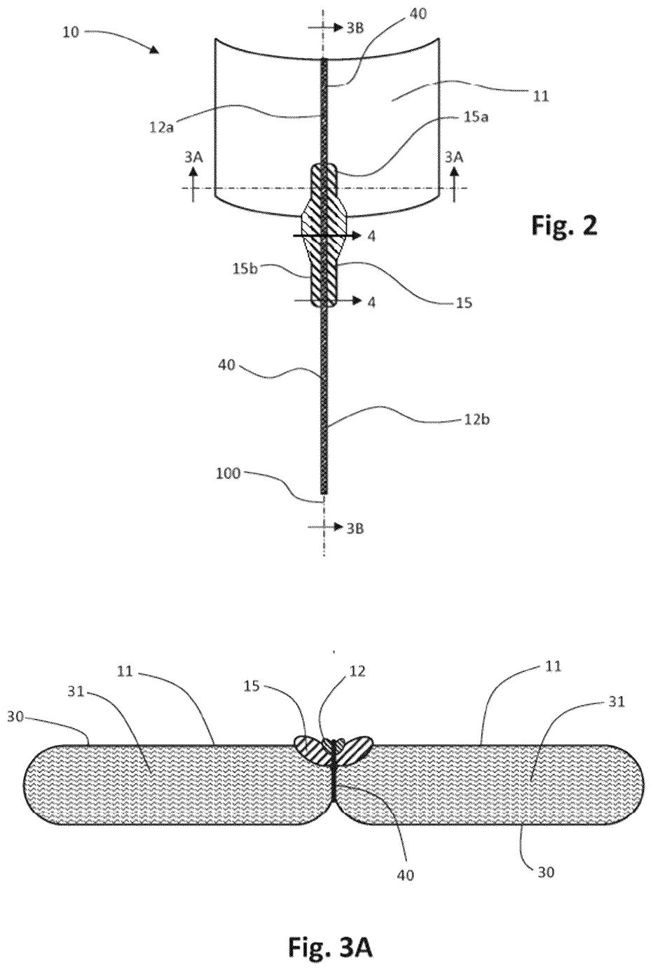

FIG. 1 illustrates one non-limiting example of an absorbent tampon 10 having a longitudinal axis 100. The absorbent tampon 10 comprises a primary absorbent 11 (which may be referred to here as a pledget), a secondary absorbent 15, and a withdrawal member 12. The primary absorbent 11 has a forward end 16 and a rearward end 17, which is opposite the forward end 16. The withdrawal member 12 may be joined to the primary absorbent member such that a leading portion 12a of the withdrawal member 12 is joined to the primary absorbent 11 and a trailing portion 12b of the withdrawal member 12 extends rearward from a location proximate the rearward end 17 of the primary absorbent 11, such as illustrated in FIG. 2. The secondary absorbent 15 may be joined to at least one of the primary absorbent 11 and the withdrawal member 12. The secondary absorbent 15 comprises a leading portion 15a positioned between the forward end 16 and the rearward end 17 of the primary absorbent 11 or adjacent to the rearward end 17 of the primary absorbent 11. The secondary absorbent 15 comprises a trialing portion 15b that is opposite the leading portion 15a and is extends rearward from a location proximate the rearward end 17 of the primary absorbent 11. Stated another way, the trialing portion 15b of the secondary absorbent is that portion of the secondary absorbent beginning external to the rearward end of the primary absorbent to the end of the secondary absorbent, or that portion of the secondary absorbent that is external to the primary absorbent. The secondary absorbent may be structured such that the material is tapered. For example, the trailing portion 15b may be tapered such that more material, a greater mass of material, is present in the area adjacent the rearward end 17 of the primary absorbent and the mass of material may be reduced as the trailing portion 15b extends away from the rearward end 17. The trailing portion 15b of the secondary absorbent 15 may be positioned between the leading portion 12a of the withdrawal member 12 and the trailing portion 12b of the withdrawal member 12. The withdrawal member 12 is configured to extend beyond the trailing portion 15b of the secondary absorbent 15 so that the user is able to easily access the tampon product for removal. Tampons contemplated herein, however, are not limited to structures having the particular configuration shown in the figures.

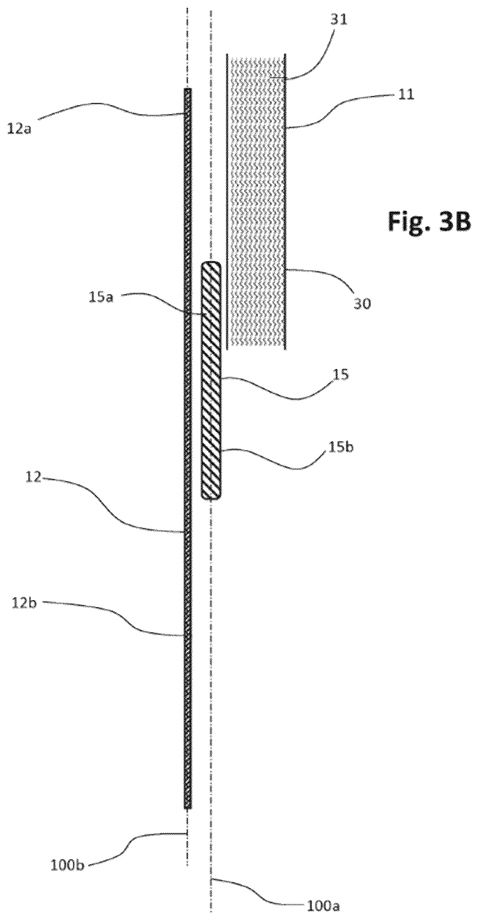

The primary absorbent 11 of the tampon 10 as shown in FIG. 1 has a forward end 16 and a rearward end 17. During manufacture of the tampons the primary absorbent 11 may be folded, bunched, compressed, and/or otherwise formed in size and shape, from its initially manufactured configuration (e.g. as shown in FIGS. 2 and 3A) into a generally cylindrical configuration (e.g. as shown in FIG. 1) in the radial direction, the longitudinal direction, or in both the radial and longitudinal directions. While the primary absorbent 11 may be formed into a substantially cylindrical configuration, such as illustrated in FIG. 1, other shapes are also possible. These may include shapes having a cross section which may be described as oval, elliptical, ovoid, stadium, rectangular, triangular, trapezoidal, semi-circular, or other suitable shapes. The primary absorbent contemplated herein may have any suitable form and structure, for example, as depicted in FIGS. 2 and 3A. Other non-limiting examples of suitable primary absorbent form, material composition, and structure are depicted and described in US2010/0268182 and US2007/0260211, which are incorporated by reference.

Prior to formation into a self-sustaining form, the primary absorbent 11 may be of any suitable shape, size, material, or configuration. In the non-limiting example shown in FIGS. 2, 3A and 3B, primary absorbent 11 includes a batt or other mass of absorbent material 31, disposed within an outer wrapper 30, also referred to herein as an overwrap. This type of primary absorbent may be formed on a continuous processing line wherein absorbent fibrous material is continuously deposited (e.g., via an airlaying process) to form a continuous batt having a desired cross-direction width and depth/weight, on a continuous web of wrapper material being conveyed along a machine direction. The wrapper material web may then be wrapped about the batt by suitable web guiding equipment, and affixed to itself via, e.g., adhesive, to form a continuous wrapped batt. Individual primary absorbents may then be cut from the continuous batt by repetitive die cutting across the moving batt (i.e., cutting along the cross direction). The cross-direction cuts may be linear, which will result in rectangular primary absorbent. Alternatively, the cross-direction cuts may be non-linear; in the example depicted, the cutting tool may be configured to make cuts forming the respective forward and rearward ends of each successive primary absorbent, having an arched or curved profile. With reference to FIG. 2, for a primary absorbent of the configuration depicted, this curved profile, or in a slight modification, a cut profile that will impart the uncompressed primary absorbent with a chevron shape, helps facilitate subsequent compression and formation into a cylindrical or capsule-shaped form with rounded or otherwise tapered forward and rearward ends, through the graduating reduction or tapering down, via the cut profile, in the bulk/quantity of material that must be compressed at each end. Other shapes that embody a tapering down of the quantity of material present toward the forward and rearward ends of the primary absorbent are also contemplated.

While the primary absorbent 11 shown in FIG. 2 is approximately chevron-shaped, other shapes such as but not limited to rectangular, trapezoidal, triangular and hemispherical may be used for tampons within contemplation of the present disclosure. It may be desired, however, that the cut profile be configured to form respective rear and front cut ends of respectively leading and trailing primary absorbents being cut from the batt as it moves through the manufacturing line, with no generation of cutoff waste/scrap. It is to be appreciated that the non-limiting example of an end cut profile reflected in FIG. 2 provides this benefit.

In other examples (not specifically shown), the primary absorbent 11 may be a laminar structure including integral or discrete layers. As noted, in the example shown in FIGS. 3A and 3B, the primary absorbent 11 may include an overwrap 30 and one or more layers of absorbent material 31 positioned within the wrapper or the wrapper may be disposed on at least a portion of the absorbent material 31 of the primary absorbent 11. In other examples, the primary absorbent need not have a layered structure at all. The primary absorbent prior to compression may have an uncompressed thickness of from about 6.0 mm to about 8.5 mm. The size and shape of the primary absorbent prior to compression may vary according to the size of the absorbent tampon. A tampon having a light absorbency (a light tampon has an absorbent capacity as measured by the standard syngyna test of less than 6 grams) may include a primary absorbent prior to compression of from about 6.0 mm to about 6.8 mm. A medium absorbency tampon (a medium tampon—also referred to as a regular tampon—has an absorbent capacity as measured by the standard syngyna test of from 6 grams to 9 grams) may include a primary absorbent prior to compression of from about 6.8 mm to about 7.5 mm. A heavy absorbency tampon (a heavy tampon—also referred to as a super tampon—has an absorbent capacity as measured by the standard syngyna test of from 9 grams to 12 grams) may include a primary absorbent prior to compression of from about 7.5 mm to about 8.5 mm. The greater the uncompressed thickness of the primary absorbent, the greater the void volume when the primary absorbent is compressed, such as illustrated in FIG. 1. However, the uncompressed thickness of the primary absorbent cannot become too large or the absorbent tampon no longer becomes usable by consumers. The aforementioned ranges of the uncompressed thickness of the primary absorbent for the various sizes have been found to have increased void volume while still maintaining the consumer preferred size of the primary absorbent when compressed. To facilitate compression into its self-sustaining form the primary absorbent 11 may be folded, such as described herein, may be rolled (e.g. as in currently marketed U BY KOTEX brand tampons, a product of Kimberly-Clark Worldwide, Inc., Irving, TX), may comprise a “petal” structure (e.g. of overlaying/underlaying, crossing rectangular patches of absorbent material, in a configuration present in PLAYTEX SPORT brand tampons, a product of Edgewell Personal Care LLC, Chesterfield, MO) or any other of the structures and configurations which are known in the art relating to tampon primary absorbents and their manufacture.

The primary absorbent 11 comprises absorbent material 31. The absorbent material 31 therein may include a wide variety of liquid-absorbing materials commonly used for absorbency in absorbent articles, such as rayon fibers, cotton fibers, and/or comminuted wood pulp fibers (sometimes called “airfelt”). Examples of other suitable absorbent materials may include creped cellulose wadding; spun and/or meltblown polymer fibers or filaments; chemically stiffened, modified or cross-linked cellulosic fibers; other synthetic fibers such as polyamide fibers (e.g., nylon fibers); peat moss; absorbent foams (such as open-celled foam formed through polymerization of a high internal phase water-in-oil emulsion); nonwoven web materials of natural and/or synthetic fibers or combinations thereof, tissue including tissue wraps and tissue laminates; or any equivalent material or combinations of materials, or blends or combinations of these. Suitable rayon fibers may include but are not limited to viscose, MODAL, TENCEL (or lyocell). Rayon fibers may be tri-lobal and conventional rayon fibers. Rayon fibers may at least one of a round, trilobal, ellipsoid, stadium, and hollow cross sectional shape. It is to be appreciated that round fibers include those fibers with a perfectly circular cross sectional area and fibers that are substantially round with some minor deviation in the absolute radius as the fiber is assessed about the radius across the 360 degrees of the cross sectional shape. Rayon fibers may be needle punched rayon. Suitable cotton fibers may include long fiber cotton, short fiber cotton, cotton linters, T-fiber cotton, card strips, and comber cotton. Preferably, the cotton fibers or fabric layer thereof should be scoured (for removal of natural hydrophobic waxes and impurities) and bleached (for whiteness) and may be imparted with a glycerin finish (for enhancing compaction), a leomin finish (for lubricity), or other suitable finish. In some embodiments, it may be desired that rayon or cotton or a blend thereof, forms the greater proportion (by weight) of the absorbent material 31, or that rayon alone forms the greater proportion (by weight) of the absorbent material 31, since rayon fibers may possess absorbency properties or capacity greater than those of other fibrous materials, per unit weight and/or per unit cost. For example, the primary absorbent may comprise greater than 50% by weight rayon fibers and less than about 50% by weight cotton fibers. More preferably, the primary absorbent may comprise greater than 80% by weight rayon fibers and less than about 20% by weight cotton fibers. The primary absorbent may comprise about 95% by weight rayon fibers and about 5% by weight of cotton fibers. The primary absorbent may comprise about 90% by weight rayon fibers and about 10% by weight of cotton fibers. The primary absorbent may comprise more than 95% by weight rayon and less than 5% by weight of cotton fibers. Additionally, superabsorbent materials, such as superabsorbent polymers or absorbent gelling materials may be incorporated into the primary absorbent.

In some embodiments, the primary absorbent 11 may be formed of a body of soft absorbent fibrous material such as rayon fibers or cotton fibers or a combination or blend thereof, and the wrapper 30 may be formed of a woven, knitted or nonwoven web fabric material of suitable composition. The materials for the body may have the form of nonwoven or woven fabric or a batt formed by any suitable process such as airlaying, carding, wetlaying, hydroentangling, or other known fiber deposition and consolidation techniques.

The absorbent material of the primary absorbent 11 may be surrounded with a liquid permeable overwrap 30. Overwrap materials and processes may include rayon, cotton, spunlace, wetlaid, needlepunched, hydroentangled, adhesive bonded, thermal bonded, pressure bonded, spunbond monocomponent, bicomponent or multicomponent fibers, or other suitable natural or synthetic fibers known in the art. If the primary absorbent 11 is layered, the layers may include different materials. For example, the wrapper 30, may include fibers comprising primarily of rayon, while the absorbent material 31 may include fibers comprised primarily of cotton. In other examples the wrapper may comprise primarily cotton, and the intermediate layer or layers may comprise primarily rayon. Optionally, the entire primary absorbent 11 may be formed of a uniform or nonuniform blend of materials throughout. In some embodiments, wrapper 30 may be formed of a nonwoven web of spunbond fibers. The spunbond fibers may be spun from, for example, polymer resin including polyolefins such as polypropylene, polyethylene, or a blend or combination thereof. In a more particular embodiment the spunbond fibers may be spun bicomponent fibers including a first polypropylene resin component and a second differing polypropylene resin component or a polyethylene resin component. When formed of ordinarily hydrophobic materials such as polyolefins (including polypropylene and polyethylene) wrapper 30 material may be treated, e.g., by application of a suitable surfactant, to render it hydrophilic, so that it will readily attract and permit aqueous fluid to wick therethrough to the absorbent material within the wrapper. A nonwoven web material formed of polymeric material as described may be desired to form the wrapper, over natural fibrous materials or semi-synthetic rayon, for reasons of having a soft, smooth and comfortable feel and low friction against sensitive skin and internal tissues, relatively low cost, and superior wet structural integrity.

The primary absorbent 11 may have any suitable size, shape, and thickness that will both provide a suitable quantity of absorbent material and resulting absorption capacity, while permitting compression into a self-sustaining form of a size and shape suitable for easy and comfortable insertion. An uncompressed, opened size similar to those of conventional currently available tampons has been found to work well. A typical size for an uncompressed primary absorbent may be from about 2 cm to about 8 cm in longitudinal length and from about 3 cm to about 8 cm in lateral width, including any combination of length and width within those ranges, in combination with an uncompressed thickness anywhere from about 1 cm to about 3 cm. Total basis weight for a flat, uncompressed and open primary absorbent, may be from about 150 g/m² to about 1,400 g/m², calculated as the weight of the primary absorbent divided by the largest surface area on one side of the primary absorbent. Optionally, a primary absorbent 11 that is shorter and wider than the ranges given above may also be desired in some circumstances to promote relatively greater swelling/expansion in a lateral or radial direction during use.

The primary absorbent of the present invention may be compressed into a generally cylindrical, self-sustaining form in the width direction, the radial direction, the axial direction, or any combination of these directions. Once compressed (shown in FIG. 1), the primary absorbent may be inserted into an applicator. For the sake of clarity, the compressed primary absorbent may be referred to as the absorbent tampon 10 (shown in FIG. 1). The compressed primary absorbent may have a compressed tampon length measured parallel to and along the longitudinal axis from the forward end to the rearward end of from about 30 mm to about 49 mm or from about 31 mm to about 51 mm. More specifically, a light absorbency, or light tampon, may have a compressed tampon length of from about 30 mm to about 38 mm. A medium absorbency, or regular tampon, may have a compressed tampon length of from about 39 mm to about 44 mm. A high absorbency, or super tampon, may have a compressed tampon length of from about 45 mm to about 49 mm. Once inserted into the vagina, the absorbent tampon 10 (shown in FIG. 1) expands upon fluid contact. In order to reduce the likelihood of leakage, the absorbent tampon should expand as much as possible. Unfortunately, the level of compression of the pledget can negatively impact the amount of expansion that an absorbent tampon can exhibit. Additionally, the amount of material and selection of fibers that form the primary absorbent impact the amount of expansion.

It has been thought that a combination of rayon fibers and cotton fibers, such as previously discussed herein, allows for the creation of high void volume and sufficient expansion of the primary absorbent when contacted by fluid. Traditionally, trilobal rayon fibers have been used because their shape allows for a generally smaller mass of material of the primary absorbent to deliver a target syngyna absorbency while having an improved expansion of the primary absorbent during use. However, it has been found that using a combination of round rayon fiber and cotton fiber and not increasing the dry mass of the primary absorbent material, the syngyna absorbency will be reduced while the expansion of the primary absorbent during use may be maintained. Table 1 includes both inventive and comparative examples supporting this determination.

TABLE 1

| | Inventive | Inventive | Conventional | Conventional | Conventional | Conventional |
|---|---|---|---|---|---|---|
| | Example 1 | Example 2 | Example 1 | Example 2 | Example 3 | Example 4 |
| Details of the | Super | Super | Lighttime_GC_ | Tmaxx_GC_ | Chinese | P&G, |
| Absorbent | Absorbency | Absorbency | China_Super | China_Super | Fiber_GC_ | TAMPAX |
| Tampon | | | Absorbency_ | Absorbency | China_Super_ | PEARL, Super |
| | | | LOT 40522T11 | LOT | Absorbency | Absorbency, |
| | | | BD | 42692T11 BD | LOT | Lot # |
| | | | | | 43062T12 BY | 4246243026- |
| | | | | | S | 69 |
| Primary | Round rayon | Round rayon | Round Rayon | Cotton/Round | Round Rayon | Trilobal |
| Absorbent Fiber | 95% | 95% | | Rayon mix | | 90% |
| Composition | Cotton 5% | Cotton 5% | | | | Cotton 10% |
| (by weight) | | | | | | |
| Dry Tampon | 2.60 | 3.0 | 2.63 | 2.76 | 2.99 | 2.60 |
| Mass (g)-A | | | | | | |
| Syngyna (g)-B | 9.9 | 10.6 | 9.7 | 10.1 | 9.7 | 10.8 |
| Tampon | 3.8 | 3.6 | 3.7 | 3.7 | 3.3 | 4.2 |
| Efficiency | | | | | | |
| (g/g)-B/A | | | | | | |
| Expansion | 20.9 | 22 | 19.5 | 19.4 | 19.4 | 21.4 |
| Width (mm) | | | | | | |
| Tampon | 0.3 | 0.33 | 0.30 | 0.35 | 0.37 | 0.27 |
| Density (g/ml) | | | | | | |

As included in Table 1, the inventive examples and the comparative examples have the same absorbency, super absorbency, and have a syngyna absorbency withing the range of those products deemed super absorbency and having an absorbency of between 9 to 12 grams. Additionally, each of the inventive examples and comparative examples have a primary absorbent including rayon or rayon and cotton. As evidenced by the data in Table 1, Inventive Example 1, includes a primary absorbent made from round rayon fibers and cotton fibers and has a Dry Tampon Mass that is less than each of Comparative Examples 1-3, which also all include round rayon fibers. However, despite that Example 1 has a lower Dry Tampon Mass, Example 1 has a greater widthwise dynamic expansion, also referred to herein as the expansion width, as determined by the Dynamic Expansion test method disclosed herein. It is understood that the greater the expanded width, the better the primary absorbent intercepts fluid and prevents leakage. Additionally, Example 2 includes a primary absorbent made from round rayon and cotton fibers but having a larger Dry Tampon Mass. However, having this larger Dry Tampon Mass resulted in an even greater widthwise dynamic expansion, as determined by the Dynamic Expansion test method disclosed herein. Thus, Examples 1 and 2, having similar types of fibers to Comparative Examples 1-3, provide greater expansion width and syngyna absorbency resulting in a tampon that has better leakage protection.

Still referring to Table 1, Comparative Example 4 and Inventive Examples 1 and 2 include different shaped rayon fibers. Generally, it has been thought that trilobal fibers allow for lower Dry Tampon Mass and achieve greater widthwise dynamic expansion due to the trilobal shape of the fiber. However, as evidenced by the data in Table 1, Examples 1 and 2 achieved similar expansion width as Comparative Example 4 while having a similar Dry Tampon Mass and round rayon fibers.

In addition to the expansion of the absorbent tampons described herein, the absorbent tampons are housed within applicators that are not increased in diameter over what is currently available. In some forms, the insertion portion of the applicator has an outer diameter of 14 mm or less for a regular absorbency tampon and 16 mm or less for a super absorbency tampon. To fit within the applicator and for case of use, the primary absorbent may be compressed to a density ranging from about 0.25 to about 0.45 grams per cubic centimeter. The method for determining tampon density is provided below in the Test Methods section. In some forms, the pledgets are compressed to a density of greater than 0.26 grams per cubic centimeter, or a density of greater than 0.30 grams per cubic centimeter. To maintain this compressed form, the pledget, or primary absorbent may undergo setting.

The setting of the compressed pledget, also referred to herein as a primary absorbent, is believed to play a critical role in the expansion profile of the tampon pledget. Compressed tampon pledgets should hold their shape within the tampon applicator so that the tampon can be expelled from the applicator and allow for the pledget, or primary absorbent, to expand once contacted with fluid. To facilitate this, heat-setting is utilized. Some suitable methods of setting or stabilizing the tampon size and shape include heating a compressed pledget via microwaving as disclosed in U.S. patent application Ser. Nos. 15/418,032 and 15/159,316. Preferably, the compressed pledget is subject to a conditioning via a microwave source for up to 25 seconds. The power of the microwave can be 2000-6000 Watts. Forms of the present invention are also contemplated where the compressed absorbent pledgets are subjected to steam or thermal gradient conduction as described in U.S. Pat. No. 7,047,608. Additional forms are contemplated where a heated gas or other medium can be applied to the compressed pledget via at least one pore or fluid communication passage while the compressed pledget is within a closed compression mold cavity.

A withdrawal member 12, such as illustrated in FIGS. 1 and 2, is preferably joined to the primary absorbent to facilitate withdrawal of the tampon from the vagina following a desired duration of use. The withdrawal member 12 may include a leading portion 12a joined to the primary absorbent 11 and a trailing portion 12b extending beyond the rearward end 17 thereof. In other examples, the withdrawal member may be integral with the primary absorbent, or an extension of a structural component of the primary absorbent, such as of an overwrap as described above. In some examples the withdrawal member 12 may be integral with the secondary absorbent 15.

The withdrawal member 12 may be a separate section of cord, string, yarn, ribbon, knitted cord, or strip of woven or nonwoven fabric formed separately of the components of the primary absorbent and secondary absorbent, and then joined by any suitable mechanism to the primary absorbent and/or to the secondary absorbent. The joining may include sewing, adhesive attachment, thermal or pressure bonding, through-punching, penetration and/or looping of the withdrawal member about structure(s) of the primary absorbent or portions thereof, or any combination of these. A leading portion 12a of the withdrawal member 12 may be joined to any suitable location on the primary absorbent 11, although it may be preferable that the joining location be substantially laterally centered on the primary absorbent and proximate to, or include a location proximate to, the rearward end 17 of the primary absorbent, so that tensile withdrawal force in the withdrawal member, exerted by the user, acts predominately on the rearward end of the primary absorbent and does not tend to substantially rotate or reorient the primary absorbent within the user's body during withdrawal.

In some embodiments, such as illustrated in FIGS. 2, 3A, and 3B, a leading portion 12a of the withdrawal member 12 is joined to the primary absorbent 11 along a length of the primary absorbent 11, and trailing portion 12b trails free (is un-joined or unattached to the primary absorbent) beyond the rearward end 17 of the primary absorbent 11. The withdrawal member 12 may be attached to the tampon primary absorbent 11 while the primary absorbent 11 is still uncompressed, such as illustrated in FIG. 2. The withdrawal member 12 may be joined along a portion of the entire length of the primary absorbent or substantially the entire length of one major surface of the primary absorbent 11.

To minimize chances of failure of the attachment between the withdrawal member 12 and the primary absorbent (i.e., separation) during withdrawal, it may be desired that the withdrawal member be directly or indirectly joined along substantially the entire length of the primary absorbent, thereby diffusing tensile withdrawal force exerted by the user, by distributing it over the length of the primary absorbent. To further minimize chances of failure of the attachment, it may be desired that the joining of the withdrawal member and the primary absorbent include a longitudinal line of stitches that penetrate the withdrawal member 12 and the primary absorbent, thereby connecting and affixing the withdrawal member through a substantial portion of the structure of the primary absorbent, rather than only to an outer surface thereof. It is to be appreciated that the stitches may entirely penetrate (through both sides) the withdrawal member 12 and the primary absorbent 11. Such joining further diffuses withdrawal force through the body/structure of the primary absorbent. In some embodiments, the stitching may be lockstitching. In other examples, a length of the withdrawal member may be threaded through a portion of the body/structure of the primary absorbent (e.g., through a hole punched therethrough), looped around and doubled to create a pair of trailing portions. In still other examples, a length of withdrawal member may be looped around a substantial portion of the primary absorbent body without punching, and doubled to create a pair of trailing portions. The trailing portions may be tied and knotted or otherwise affixed together.

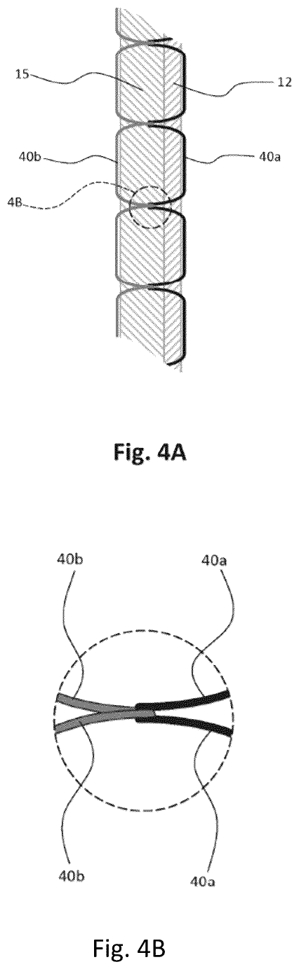

In some embodiments, lockstitching may be used to join the withdrawal member 12 and the primary absorbent 11. Herein, “lockstitching” means a line of stitches formed of at least two strands of thread disposed on opposing sides of the body(ies) to be stitched together, wherein stitches are sequentially formed as each thread meets and loops around the other, via passage through the body(ies) by one or both threads, at suitable intervals corresponding to the desired size of the stitch. In some examples, a first thread may be sequentially passed through the body(ies) to meet the second thread via use of an appropriate sewing needle, while the second thread is looped about the first thread by operation of a looper. Chainstitching consisting of two threads as described above is included within the definition. A non-limiting example of lockstitching may be seen in FIGS. 4A and 4B, depicting a longitudinal cross section through a secondary absorbent 15 and withdrawal member 12, wherein these two components are held together by a longitudinal line of lockstitching with stitches formed by front thread 40a and rear thread 40b. FIGS. 4A and 4B depict ISO #301 type lockstitching, as specified by the International Organization for Standardization, ISO 4519:1991, as an example. Other types of lockstitching may be preferred in some circumstances, for example, ISO #401 type chainstitching, which may further enhance the stitches' ability to resist unraveling themselves, and prevent unraveling of the stitched withdrawal member and/or secondary absorbent, at cut forward and/or rearward ends thereof. Where lockstitching is used to join the withdrawal member to the primary absorbent, in some embodiments, it may be desired that the lockstitching 40 extend longitudinally along substantially the entire length of the withdrawal member 12 (including both leading and trailing portions 12a, 12b). In examples in which the withdrawal member 12 is formed of a section of twisted, braided, or knitted strands or fibers, lockstitching that traverses substantially the entire length of the withdrawal member may be desired because the thread strands forming the stitches through the member are effectively intertwined with component fibers and/or strands of the member and can thereby function to substantially prevent the member from unraveling from its cut ends. However, it is to be appreciated that the lockstitching need not extend along the entire length of the withdrawal member 12.

The threads used to form the line of lockstitching be made of a suitably hydrophobic fiber material, or fiber material treated to be suitably hydrophobic, so that the lockstitching thread is unlikely to wick fluid along the trailing portion of the withdrawal member. In some examples the lockstitching thread may be formed of or include cotton fiber, processed or treated to be suitably hydrophobic. In some examples the lockstitching thread may be formed of or include polyester fiber (which in some formulations may be inherently somewhat hydrophobic). In some examples the lockstitching thread may be formed of or include a blend of cotton fiber and polyester fiber, wherein the cotton fiber may be processed or treated to be suitably hydrophobic.

The tampon 10 may also be provided with multiple withdrawal members 12. For example, two withdrawal members 12 may be attached down the length of the primary absorbent 11 and extend from the trailing portion of the primary absorbent thereof. In such an instance, the secondary absorbent may be un-joined to either withdrawal member or may be joined to one or both of the withdrawal members 12.

The withdrawal member 12 may be non-absorbent along at least a portion, such as in the location of such joining to the secondary member. As used herein, the term “non-absorbent” refers to a structure formed predominately of suitably hydrophobic materials such it does not tend to attract, wick, or retain any substantial quantity of fluid within its structure. In some examples it may be desired that substantially the entire withdrawal member 12 be hydrophobic, so that the withdrawal member does not wick menstrual fluid along its trailing portion 12b, potentially out to its trailing end. The materials comprising the withdrawal member may be inherently non-wettable or hydrophobic, or they may be treated to provide such properties. For example, a suitable wax may be applied to the withdrawal member 12 to decrease or eliminate wicking tendency. Other means for providing a material suitable for use as a withdrawal member 12 which is non-absorbent and/or non-wicking are known in the art. For example, U.S. Pat. No. 5,458,589 describes one such approach. However, the withdrawal member 12 need not necessarily be non-wicking along its entire length, even if a non-absorbent withdrawal member is desired. For example, it may be desirable to provide a withdrawal member 12 in which at least a portion of the member has a tendency or capability to wick deposited fluid upwardly toward the rearward end 17 of the primary absorbent and into the absorbent material thereof.

The withdrawal member 12 need not have uniform properties throughout its length. For example, the portion of the withdrawal member nearest the primary absorbent 11 may be manufactured and/or treated so as to have wicking capability, while the lower portion (i.e. furthest from the primary absorbent 11) of the withdrawal member 12 may be manufactured and/or treated so as to not have wicking capability. Other properties such as hydrophilicity/hydrophobicity, density, capillary size, width, thickness, and the like may also vary along the length of the withdrawal member 12.

The withdrawal member 12 may be formed of a strand or strands (such as two or more strands) of component yarn or thread material. In some examples the yarn or thread material may be formed of cotton fiber, cotton fiber processed or treated to be suitably hydrophobic, other natural plant-based fiber which may be processed or treated to be suitably hydrophobic, or polyester, or a combination or blend thereof. The component yarn or thread may be knitted, twisted or braided to form the withdrawal member stock. For maximized tensile strength per unit decitex of the withdrawal member, it may be desired that the component yarn or thread be of twisted or braided construction (rather than of knitted, woven, or other construction).

A secondary absorbent 15, described in greater detail below, may be joined to at least one of the withdrawal member 12 and the primary absorbent 11. This joining of the secondary absorbent may occur subsequently to compression of the primary absorbent 11 to a self-sustaining form. In some variations it may be desirable to join some or all of the secondary absorbent 15 to the primary absorbent 11, the withdrawal member 12, or both, prior to compression of the primary absorbent 11 to a self-sustaining form. In one method of making of a tampon 10, the secondary absorbent 15 may be integral with the primary absorbent 11 prior to compression of the primary absorbent. In any of the above mentioned manners of construction, the trailing portion 15b of secondary absorbent 15 is preferably not compressed with the primary absorbent 11; or, if compressed, is not compressed to the same degree as the primary absorbent 11.

The leading portion 15a of the secondary absorbent may be joined along a portion of or the entire length of the primary absorbent 11. The trailing portion 15b of the secondary absorbent extends or trails by a suitable length from the rearward end 17 of the primary absorbent. The material of each of the leading portion 15b and the trailing portion 15b may be tapered. For example, the leading portion 15a may include material that tapers from a lesser mass to a greater mass as the leading portion extends toward the rearward end 17 of the primary absorbent. In another example, the trailing portion 15b may include material that tapers from a greater mass to a lesser mass as the trailing portion 15b extends from the rearward end 17 of the primary absorbent, such as illustrated in FIGS. 1 and 2. The secondary absorbent 15 may be separate from, or joined to, the withdrawal member 12 along a portion or all of at least one of their respective lengths. As will be discussed below, the secondary absorbent 15 may be provided, following insertion of the tampon at a suitable location within the vaginal cavity, to extend rearward of the rearward end of the primary absorbent, further down the vaginal cavity toward the introitus, where it can be in position to contact menstrual fluid that may be present below the primary absorbent, and attract and wick liquidous components thereof, such as from bypass leakage, up to the primary absorbent 11. As previously discussed, the secondary absorbent as discussed herein addresses the problem of bypass leakage while maintaining wearer comfort. The inventors have unexpectedly discovered the solution is to have select absorbent material levels and ranges associated with the secondary absorbent located at select segments along the length of the trailing portion of the secondary absorbent, and for the secondary absorbent to have a select mean length and range. With such an inventive design combination, the overall tampon product may provide ideal coverage of the entire length of the vaginal cavity, especially the position at the vaginal opening and yet minimize wearing discomfort.

The secondary absorbent 12 may have any suitable total length. However, for the leading portion 15a, in order to ensure that an adequate portion of the surface area of the secondary absorbent 15 is exposed to contact with the primary absorbent (for purposes of facilitating movement of fluid from the secondary absorbent to the primary absorbent), it may be desired that the leading portion 15a have a length that is at least one-quarter of the total length of the primary absorbent, and more preferably at least one-third of the total length of the primary absorbent. In a particular example, the leading and trailing portions of the secondary absorbent 15a, 15b may be approximately equal in length. It may also be preferred that the leading portion of the secondary absorbent be affixed to the primary absorbent along a length that is at least 10 mm, or at least 15 mm, or at least 20 mm, or at least 25 mm.

For the trailing portion 15b of the secondary absorbent (the portion extending rearward of rearward end 17 of primary absorbent 11) is preferably shorter than the trailing portion of the withdrawal member 15. Additionally, and importantly, the trailing portion of the secondary absorbent should not be long enough to extend through the introitus when the tampon 10 is fully inserted and properly positioned within the vaginal cavity. This is important for the user to not experience discomfort from the secondary absorbent while the absorbent tampon is being used. Although dimensions of the vaginal cavity vary among individual users, it has been found that a desired length of the trailing portion 15b of the secondary absorbent 15 for most users should have a mean length from the primary absorbent of no greater than about 60 mm, or no greater than about 50 mm, or no greater than about 40 mm, or no greater than about 30 mm, or from about 20 mm to about 40 mm, or from about 30 mm to about 40 mm, or from about 30 mm to about 50 mm, or from about 40 mm to about 50 mm, measured according to the Off-Pad Secondary Absorbent Profile Method as described herein.

The mean length of the secondary absorbent is important to achieve a higher performing tampon when it comes to leaks, it has been found that select material levels and ranges of the secondary absorbent are needed to achieve reduced bypass leakage. These material levels and ranges provide ideal coverage of the entire length of the vaginal cavity while not interfering with the nerves and sensitive tissues proximate the vaginal opening. Current marketed products, generally have a secondary absorbent having a mean length from the primary absorbent of about 27 mm, as determined by the Off-Pad Secondary Absorbent Profile method described herein. The inventors have surprisingly found that a secondary absorbent mean length from the primary absorbent of greater than about 50 mm or greater than about 52 mm or greater than about 54 mm or greater than about 58 mm or about 54 mm or from about 50 mm to about 54 mm or from about 52 mm to about 54 mm or from about 52 mm to about 56 mm or from about 54 mm to about 60 mm or from about 56 mm to about 60 mm or from about 58 mm to about 62 mm, as determined by the Off-Pad Secondary Absorbent Profile method described herein. Further, because the vaginal cavity is different for individual users, the mean mass is controlled along the length of the secondary absorbent to ensure that the secondary absorbent absorbs relatively more of the bypass leakage and is comfortable for the wearer during use. Again, because the vaginal cavity is different for individual users, it has been found, generally, that the mean mass of the secondary absorbent that is nearest to the primary absorbent is greater than the mean mass of the secondary absorbent that is farthest from the primary absorbent. Stated another way, those segments of the secondary absorbent that are nearest to the primary absorbent have a greater mean mass than those segments of the secondary absorbent that are farthest from the primary absorbent. Additionally, those segments or segment that is farthest from the primary absorbent should be significantly smaller to maintain comfort of the tampon during use. To maintain comfort for a wide variety of consumers, the segment farthest from the primary absorbent should be small so as to not interference with the sensitive tissues and nerves present in the vaginal opening.

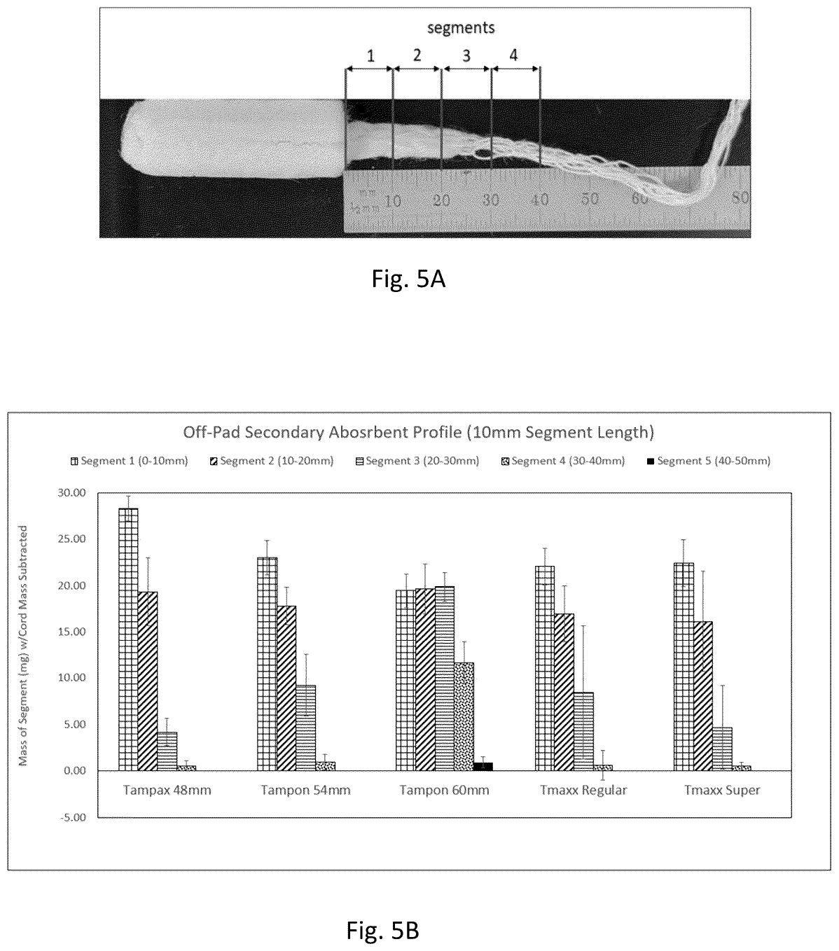

To evaluate the secondary absorbent, the secondary absorbent may be divided into segments and each segment evaluated. More specifically, the secondary absorbent may be divided into segments of 10 mm in length and the mean segment mass for each segment may be determined according to the Off-Pad Secondary Absorbent Profile method described herein. For example, a first segment may be from 0 mm-10 mm as measured along the length of the secondary absorbent from the primary absorbent. FIG. 5A illustrates a tampon having a primary absorbent, a secondary absorbent, and withdrawal member, and the secondary absorbent being divided into four segments, each of 10 mm.

With reference to FIG. 5B and Table 2 below, current market absorbent tampons and inventive samples were tested according to the Off-Pad Secondary Absorbent Profile method as described herein. The details of each of the samples tested are provided below.

Comparative sample, “Tampax 48 mm” is a currently marketed absorbent tampon product commonly known as TAMPAX PEARL™ and TAMPAX RADIANT™ including the LEAKGUARD™ Braid produced by The Procter & Gamble Company. The total length of the secondary absorbent including the leading portion and the trailing portion is about 48 mm. The secondary absorbent is made from polypropylene fibers and the withdrawal member is made from cotton fibers.

Inventive sample, “Tampon 54 mm” is an absorbent tampon having a secondary absorbent that has a total length, including the leading portion and the trailing portion, of 54 mm. The leading portion has a length of about 21 mm and the trailing portion has a length of about 33 mm. The secondary absorbent is made from polypropylene fibers and the withdrawal member is made from cotton fibers.

Inventive sample, “Tampon 60 mm” is an absorbent tampon having a secondary absorbent that has a total length, including the leading portion and the trailing portion, of 60 mm. The leading portion has a length of about 21 mm and the trailing portion has a length of about 39 mm. The secondary absorbent is made from polypropylene fibers and the withdrawal member is made from cotton fibers.

Comparative sample, “Tmaxx Regular” is an absorbent tampon product having a regular level of absorbency and a secondary absorbent, which is commonly referred to by consumers as a braid, and produced under the brand name TMAXX by Jiangsu Zhenmian Biotechnology in China.

Comparative sample, “Tmaxx Super” is an absorbent tampon product having a super level of absorbency and a secondary absorbent, which is commonly referred to by consumers as a braid, and produced under the brand name TMAXX by Jiangsu Zhenmian Biotechnology in China.

TABLE 2

| | | | | | | | | | | | Mean Length | |
|---|---|---|---|---|---|---|---|---|---|---|---|---|
| | Mean Segment Mass (mg) w/Mass | | | | | | | | | | from the | |
| | of Withdrawal Member Subtracted Out | | | | | | | | | | primary | |
| | Segment 1 | | Segment 2 | | Segment 3 | | Segment 4 | | Segment 5 | | absorbent | |
| | (0-10 mm) | | (10-20 mm) | | (20-30 mm) | | (30-40 mm) | | (40-50 mm) | | (mm) | |
| | | Std. | | Std. | | Std. | | Std. | | Std. | | Std. |
| Sample | Avg. | Dev. | Avg. | Dev. | Avg. | Dev. | Avg. | Dev. | Avg. | Dev. | Avg. | Dev. |
| Tampax | 28.3 | 1.3 | 19.3 | 3.6 | 4.2 | 1.5 | 0.6 | 0.5 | N/A | N/A | 27.3 | 0.6 |
| 48 mm | | | | | | | | | | | | |
| Tampon | 23.0 | 1.9 | 17.8 | 2.0 | 9.2 | 3.3 | 1.0 | 0.9 | N/A | N/A | 31.7 | 2.5 |
| 54 mm | | | | | | | | | | | | |
| Tampon | 19.5 | 1.8 | 19.6 | 2.7 | 19.9 | 1.6 | 11.7 | 2.2 | 1.0 | 0.6 | 42.0 | 2.0 |
| 60 mm | | | | | | | | | | | | |
| Tmaxx | 22.1 | 2.0 | 16.9 | 3.0 | 8.5 | 7.2 | 0.6 | 1.6 | N/A | N/A | 28.6 | 3.7 |
| Regular | | | | | | | | | | | | |
| Tmaxx | 22.4 | 2.5 | 16.1 | 5.5 | 4.7 | 4.5 | 0.5 | 0.5 | N/A | N/A | 26.3 | 2.1 |
| Super | | | | | | | | | | | | |

As evidenced by Table 2 and illustrated in FIG. 5B, each of the samples were tested by determining the mean length from the primary absorbent of the secondary absorbent, which may be referred to herein as the trailing portion of the secondary absorbent, and dividing the secondary absorbent into 10 mm segments and determining the mean segment mass. Each of the mean length from the primary absorbent and the mean segment mass are determined according to the Off-Pad Secondary Absorbent Profile method as described herein. Each of the samples tested includes an absorbent tampon product having an insertion end and a withdrawal end, and comprising a primary absorbent region, disposed toward the insertion end, a secondary absorbent region, and a withdrawal member.

Based on the data of Table 2, it was discovered, that an absorbent tampon product comprising a secondary absorbent having a mean length from the primary absorbent of from about 20 mm to about 40 mm, and the secondary absorbent region exhibiting a mean segment mass at a length of 20-30 mm from the primary absorbent of greater than about 9.0 mg, or from about 9.0 mg to about 30 mg, or from about 9.0 mg to about 25 mg, or from about 9.0 mg to about 20 mg or from about 9.2 mg to about 20 mg, according to the Off-Pad Secondary Absorbent Profile method as described herein, exhibits a relatively greater leakage protection while maintaining user comfort. Additionally, or alternatively, the secondary absorbent region may comprise a first segment having a length from the primary absorbent of 0 mm to 10 mm and a second segment having a length from the primary absorbent of 10 mm to 20 mm, and the difference between the mean segment mass of the first segment and the mean segment mass of the second segment is less than 5 mg or less than about 10 mg. Additionally, or alternatively, the secondary absorbent may comprise a third segment having length of 20 mm to 30 mm from the primary absorbent and a fourth segment having a length from the primary absorbent of 30 mm to 40 mm, and the difference between the mean segment mass of the third segment and the mean segment mass of the fourth segment is greater than 8 mg, or greater than about 10 mg. By relatively increasing the mean segment mass in the portion of the secondary absorbent nearer to the primary absorbent and controlling the difference between the levels of the mean segment mass along the secondary absorbent such that the mean segment mass is within a certain tolerance as related to the adjacent segment, leakage prevention is relatively increased.

In some embodiments, to have relatively higher bypass leakage protect while maintaining comfort, the secondary absorbent has a mean length from the primary absorbent of from about 20 mm to about 40 mm and comprises a first segment having a length from the primary absorbent of 0 to 10 mm, a second segment having a length from the primary absorbent of from 10 mm to 20 mm, and a third segment having a length from the primary absorbent of from 20 mm to 30 mm, and the difference between the mean segment mass of the third segment and the mean segment mass of the fourth segment is at least 8 mg or at least 10 mg or at least 12 mg. Additionally, or alternatively, the mean segment mass of the fourth segment may be at least about 10% of the Mean Segment Mass of the third segment. Additionally, or alternatively, the mean segment mass of first segment may be less than or substantially equal to or equal to the mean segment mass of the second segment. Additionally, or alternatively, the mean segment mass of the third segment is at least 80% of the mean segment mass of the second segment. Additionally, or alternatively, the secondary absorbent may comprise a fourth segment having a length from the primary absorbent of from 30 mm to 40 mm, and the mean segment mass of the fourth segment is at least 50% of the mean segment mass of the third segment. The mean segment mass of the fourth segment is less than the mean segment mass of the third segment. The mean segment mass of the fourth segment may be greater than about 10 mg or greater than about 12 mg.

Further, based on the data of Table 2, the inventors have disclosed that the consistency of the mean segment mass of the secondary absorbent is also important in maintaining expected performance, including reduced bypass leakage. Stated another way, the taper of material or difference in the mean segment mass as the secondary absorbent extends from the rearward end of the primary absorbent is important to maintain comfort and bypass leakage protection. The trailing portion 15b may taper from an area adjacent the rearward end having a greater mass of material and having a decreasing mass of material as the secondary absorbent extends from the rearward end of the primary absorbent. The amount of taper or change in mean segment mass may be controlled for optimum bypass leakage protection and comfort. For example, a secondary absorbent having a mean length from the primary absorbent of from about 20 mm to about 40 mm and having a mean segment mass at a length of 20-30 mm from the primary absorbent of greater than about 5.0 mg or from about 5.0 mg to about 30 mg or from about 5.0 mg to about 25 mg or from about 5.0 mg to about 20 mg, and a standard deviation of the mean segment mass at the length of 20-30 mm from the primary absorbent of less than about 7 mg, achieves relatively increased consistency and relatively higher leakage performance. Additionally, or alternatively, the Mean Segment Mass of the secondary absorbent at the length of 20-30 mm from the primary absorbent may be from about 9.0 mg to about 25 mg. Additionally, or alternatively, the Mean Segment Mass of the secondary absorbent at a length of 30-40 mm from the primary absorbent may be from about 1.0 mg to about 5 mg.

In some embodiments, the secondary absorbent may be made longer, such as the Tampon 60 mm, and have relatively increased bypass leakage protection while maintaining user comfort. In some embodiments, the secondary absorbent region has a mean length from the primary absorbent of from about 30 mm to about 50 mm, and the secondary absorbent region exhibits a mean segment mass at a length of 30-40 mm from the primary absorbent of from about 2.5 mg to about 20 mg, or from about 2.5 mg to about 30 mg or from about 2.6 mg to about 40 mg. Alternatively, or in addition, the secondary absorbent may comprise a first segment having a length from the primary absorbent from 0 mm to 10 mm and a second segment having a length from the primary absorbent from 10 mm to 20 mm, and the difference between the mean segment mass of the first segment and the mean segment mass of the second segment is less than 5 mg or less than about 8 mg or less than about 10 gm or less than about 12 mg. Alternatively, or in addition, the secondary absorbent region comprises a second segment at a length from the primary absorbent of from 10 mm to 20 mm, and a third segment at a length of 20 mm to 30 mm from the primary absorbent, and the difference between the mean segment mass of the second segment and the mean segment mass of the third segment may be less than 8.3 mg or less than 8 mg or less than about 7.75 mg. Alternatively, or in addition, the secondary absorbent comprises a third segment having a length of 20 mm to 30 mm from the primary absorbent and a fourth segment having a length from the primary absorbent from 30 mm to 40 mm, and the difference between the mean segment mass of the third segment and the mean segment mass of the fourth segment may be greater than 8 mg or greater than 10 mg or greater than 12 mg.

In some embodiments, the secondary absorbent has a mean length from the primary absorbent of from about 30 mm about 50 mm. The secondary absorbent region comprises a first segment having a length from the primary absorbent of from 0 mm to 10 mm and a second segment having a length from the primary absorbent from 10 mm to 20 mm. The mean segment mass of the second segment may be at least 68.5% or at least 77.25% or at least 75% or at least 80% of the mean segment mass of the first segment. Additionally, or alternatively, the secondary absorbent region may comprise a third segment having a length from the primary absorbent of from 20 mm to 30 mm and the third segment may be at least 51% or at least 52% or at least 55% or at least 60% of the mean segment mass of the second segment. Additionally, or alternatively, the secondary absorbent region may comprise a fourth segment having a length from the primary absorbent of from 30 mm to 40 mm, and the fourth segment may be at least 12% or at least 15% or at least 20% of the mean segment mass of the third segment. Additionally, or alternatively, the secondary absorbent may comprise a fifth segment having a length from the primary absorbent of from 40 mm to 50 mm, and the mean segment mass of the fifth segment is at least 5% or at least 8% or at least 10% or at least 15% or at least 20% of the mean segment mass of the fourth segment.

In some embodiments, the secondary absorbent has a mean length from the primary absorbent of from about 30 mm about 50 mm and comprises a first segment having a length from the primary absorbent of from 0 mm to 10 mm and a second segment having a length from the primary absorbent from 10 mm to 20 mm, and the mean segment mass of the second segment at least 80% or at least 85% or at least 90% of the mean segment mass of the first segment. Additionally, or alternatively, the mean segment mass of the first segment may be less than or equal to the mean segment mass of the second segment. The secondary absorbent may comprise a third segment having a length from the primary absorbent from 20 mm to 30 mm, and the mean segment mass of the third segment is at least 80% or at least 85% or at least 90% of the mean segment mass of the second segment. Additionally, or alternatively, the secondary absorbent may comprise a fourth segment having a length from the primary absorbent of from 30 mm to 40 mm, and the mean segment mass of the fourth segment may be at least 50% or at least about 60% or at least about 75% of the mean segment mass of the third segment. The mean segment mass of the fourth segment may be greater than 10 mg or greater than about 12 mg or greater than about 15 mg. The mean segment mass of the fourth segment may be less than the mean segment mass of the third segment.

As previously discussed, the increased length and/or the relatively greater mean segment mass of the secondary absorbent may be accompanied by an increase in the drag force of the secondary absorbent within the pusher of the applicator and disrupt overall smooth delivery of the absorbent tampon into the vaginal cavity. The inventors discovered the tampon products in an applicator with inventive secondary absorbents of the present invention may provide acceptable insertion experiences by having a secondary absorbent design exhibiting a pusher peak drag force of less than about 10 grams or less than about 25 grams or less than about 50 grams or less than about 70 grams, or less than about 85 grams, according to the Tampon Pusher Drag Force method described herein. Such tampon products contained within an applicator may deliver high performance insertion experience as the secondary absorbent exhibits a low enough drag force on the internal pusher wall to enable smooth comfortable expulsion of the tampon including the secondary absorbent out of the applicator barrel and pusher.

The secondary absorbent 15 may be formed of a suitable configuration of fibrous material having suitable fluid handling properties and tensile strength. It may be desired that the secondary absorbent be formed separately of the withdrawal member, and of one or more material(s) distinct from the one or more materials forming the withdrawal member. As discussed above, in some examples it may be desired that the material(s) forming the withdrawal member be suitably hydrophobic, to reduce or avoid wicking of fluid along the withdrawal member. Thus, the secondary absorbent region may be more hydrophilic than the withdrawal member to help prevent leakage. It may be desired that hydrophobic fibrous components of the withdrawal member not be present within the structure of the secondary absorbent, where they can contribute to obstructing or interrupting wicking. The secondary absorbent member may be joined to or separate from the withdrawal member. The secondary absorbent member and the withdrawal member may be coaxial or may not be coaxial. Stated another way, the mass of the secondary absorbent member may be substantially axially centered about the withdrawal member or the mass of the mass of the secondary absorbent member may not be axially centered about the withdrawal member. However, it may be desired that the respective structures of the secondary absorbent and withdrawal member not be coaxial and/or not intermingled. As illustrated in the figures, the secondary absorbent and the withdrawal member may be arranged in contact with each other (or not) along a substantially parallel, non-coaxial configuration; see, e.g., FIG. 3B, depicting such a non-coaxial configuration, where the parallel longitudinal axes 100a, 100b of the secondary absorbent 15 and withdrawal member 12, respectively, are not the same. Tampons of the type and configurations contemplated herein may also have or include any combination of features described in U.S. Pat. No. 11,779,494 and/or U.S. Patent Publication No. 2020/0188190.

The ability and tendency of a fibrous structure to draw in and transport (herein, “wick”) aqueous fluid against external forces acting on the fluid (such as gravity) is a function of several features of the structure. These include the mean segment mass of the individual segments, the extent of hydrophilicity of the surfaces of the fibers; the extent of capillarity within the structure (where capillarity relates to the number and average size and volume of interstitial spaces constituting potential fluid passageways between and among the fibers, resulting from the extent and manner of fiber consolidation in the structure); the complexity of the fibers' surface geometry (ies); and the extent to which the structure has already drawn in and retains (i.e., has absorbed) fluid. Capillarity of a fibrous structure relates to the amount of fiber surface area that is present within the structure, per unit volume of the overall structure, and to the density of consolidation of the fibers in the structure, which affects the size and volume of the interstitial spaces or fluid passageways. The size and volume of the interstitial passageways affects the degree to which the aggregate attractive pull of the hydrophilic fiber surfaces in contact with the fluid can overcome forces that resist it, i.e., surface tension of the fluid and external forces such as, e.g., gravity or pressure differential. For example, for a structure formed of a given fiber composition, interstitial passageways which are too large can make the structure ineffective at wicking upward against gravitational pull because there is an insufficient aggregate area of hydrophilic fiber surfaces in contact with the fluid to create attractive pull sufficient to overcome gravitational pull acting on the relatively large fluid volume and mass in the relatively large passageways, and surface tension of the fluid mass itself tending to resist separation into smaller fluid volumes. On the other hand, interstitial passageways which are too small and/or insufficient in aggregate volume such that, while effective at moving fluid in small volume, can be physically restrictive with respect to wicking volume flow rate. For a given type of hydrophilic fibers, there will be an optimum capillarity in a structure formed of them, at which wicking potential is maximized.

Thus, it may be desired that the component fibrous material of the secondary absorbent 15 have a combination of fibers with hydrophilic surface properties, and optimized capillarity, to promote wicking of fluid therealong, but at the same time, not have, or not be assembled in a configuration having, a combination of capillarity and hydrophilicity that render it more likely to attract and retain menstrual fluid against the wicking potential of the material(s) of the primary absorbent 11. It is preferred that the material of the secondary absorbent serve to wick fluid to the primary absorbent, but that the material(s) of the primary absorbent have greater wicking potential and absorbency so as to be effective at drawing menstrual fluid from the secondary absorbent over the expected normal duration of use of the tampon.

The secondary absorbent may be formed of fibrous material(s) similar to those used to form the primary absorbent. The secondary absorbent may be formed from polyester fibers, polyethylene fibers, polypropylene fibers, cotton fibers, rayon fibers, including viscose fibers and lyocell fibers, cotton fibers, or any combination thereof. In such examples the fibrous material forming the secondary absorbent should be configured such that the body of the secondary absorbent has less wicking potential than the primary absorbent. The wicking potential of the secondary absorbent can be adjusted by the selection of material of which it is formed, for its relative level of fiber surface hydrophilicity and its relative capillarity. Capillarity may be adjusted by the manner in which the fibers forming the secondary absorbent are consolidated and densified within in the structure.

While a tampon with a secondary absorbent may absorb some menstrual fluid into the secondary absorbent and may even wick fluid to the primary absorbent to some extent, it is believed from research that the effectiveness of the combination may not be meaningfully noticeable to a user unless its ability to capture and wick fluid through the secondary absorbent up to the primary absorbent exceeds a particular value for Wicking as set forth and described herein. A combination of materials described herein, used to constitute and configure a tampon product, may be selected and assembled as described to provide a tampon that will wick at least 1.2 grams, more preferably at least 1.5 grams and even more preferably at least 1.8 grams of test fluid up through the secondary absorbent as measured using the Wicking Measurement method herein. Using combinations of materials described herein, the inventors have achieved measured wicking as high as 2.3 grams, although it is contemplated that greater levels as high as 3 grams, 4 grams or even 5 grams may be achievable through experimentation, using suitable combinations of materials and configurations identified herein or otherwise known to the person of ordinary skill in the art. The ability of the primary absorbent to draw fluid from the secondary absorbent may be further enhanced by processes and configurations described in U.S. U.S. Patent Publication No. 2019/0374405 and U.S. Pat. No. 11,925,535, which are incorporated by reference herein.

The secondary absorbent may have the form as previously discussed, such as a strip of nonwoven, woven or knitted cloth, or ribbon. However, for purposes of maximizing the presence of generally longitudinally-oriented fluid pathways for wicking fluid to the primary absorbent, it may be desired that the fibers forming the secondary absorbent are predominately longitudinally oriented or biased, and held together by twisting (in the manner in which twisted yarn or rope is formed), or by braiding, rolling, knitting, entangling, twisting, or weaving. A braided or twisted configuration, in combination with substantially directionally-aligned carded and/or tow fibers, may help increase the presence, number and length of generally longitudinal pathways through and along the fibrous material forming the secondary absorbent, along which fluid may travel more directly along hydrophilic surfaces thereof toward the primary absorbent.

To enhance unitized structural integrity of the tampon, the secondary absorbent is joined to a portion of the primary absorbent. The leading portion of the secondary absorbent may be joined to the primary absorbent and the trailing portion of the secondary absorbent may extend beyond the rearward end, unattached to the primary absorbent. In some embodiments, the leading portion of the secondary absorbent may be stitched to the primary absorbent and a portion of the withdrawal member may be stitched to the primary absorbent. The secondary absorbent 15 and/or the withdrawal member may be lockstitched to the primary absorbent member. In some examples the withdrawal member 12 and the secondary absorbent 15 may be joined together to the primary absorbent, via the same configuration/line of lockstitching. In such configuration, it may be desired that the secondary absorbent be disposed in direct contact with the primary absorbent, and preferably that the secondary absorbent be disposed between the primary absorbent and with the withdrawal member, to provide for direct contact and fluid transfer between the secondary absorbent and the primary absorbent, unobstructed by the (e.g., hydrophobic) structure of the withdrawal member. In some examples, however, the secondary absorbent 15 may be joined to the primary absorbent 11 by a mechanism differing from that joining the withdrawal member 12 to the primary absorbent 11 and may also be physically separated from the withdrawal member 12 at or along location(s) of attachment on the primary absorbent. In some examples, the secondary absorbent 15 may be attached to the primary absorbent 11 by adhesive bonds, by thermal compression or ultrasonic bonds (in which respective material(s) of the secondary absorbent and of the primary absorbent are fused or welded together) or may be stitched to the primary absorbent by stitches separate from stitches attaching the withdrawal member to the primary absorbent 11.

In some circumstances, it may be desired that a rearward-most trailing portion of the secondary absorbent 15 have a free length including a free end that is not stitched or otherwise affixed to the withdrawal member 12. This may further improve the ability of the secondary absorbent 15 to capture and wick fluid to, into and/or through, its trailing portion 15b.

One or more threads used to stitch the secondary absorbent 15 to the primary absorbent 11 may be selected for suitable tensile strength and hydrophobicity such that they may be extended beyond the trailing rearward end of the secondary absorbent by a suitable length, and by themselves serve as the withdrawal member. This configuration eliminates the need for, and expense and complexity associated with including, a separate withdrawal member. Where two or more stitching threads are used to lockstitch the secondary absorbent to the primary absorbent, they may be twisted, braided, or otherwise suitably intertwined, entangled, or combined into a singularized member configuration in a trailing portion extending rearwardly from the secondary absorbent.

It may be desired that the material used to form the secondary absorbent be tinted or pigmented to impart the secondary absorbent with a color that visibly contrasts with the color(s) of the materials forming the primary absorbent and/or withdrawal member. This may be deemed useful for visually signaling to the user that a differing material is present in the secondary absorbent, suggesting a functionality distinct from that of the withdrawal member. In connection with appropriate information on, or associated with, packaging for the tampon product, such tinting or pigmenting can advantageously serve to remind the user that the secondary absorbent is present to provide supplemental protection against leakage. When the primary absorbent and/or materials constituting the primary absorbent have a substantially white color (which is, for example, the natural color for suitably processed, undyed cotton, and is believed to be preferred by many consumers because it connotes purity, cleanliness, sanitation and/or freshness), it may be desired that the material(s) constituting the secondary absorbent are imparted with a non-white color that not only visibly contrasts with the color of the primary absorbent, but also visibly contrasts with color(s) of areas of a primary absorbent as stained by menstrual fluid as it may appear immediately following use. Thus, in some examples it may be preferred that the non-white color of the secondary absorbent be selected from a range of colors that will visibly contrast with the color of the primary absorbent, and with the color(s) of the primary absorbent when stained by menstrual fluid, as it appears immediately following withdrawal of the tampon. This coloration feature may serve to provide the user with additional signal of the functionality of the secondary absorbent. For purposes herein, “substantially white” means having CIE L*a*b* values when measured according to the Color Measurement Method set forth below, in which L* is ≥87, and the absolute values of each of a* and b* are ≤2. A secondary absorbent color that visibly contrasts with the color of the primary absorbent is any color that exhibits a ΔE*≥15 from the color of the primary absorbent, measured according to the Color Measurement Method below. Techniques for imparting varying colors, and adjusting the depth thereof, to synthetic, semi-synthetic and natural plant-based fibers or filaments, or materials made therefrom (e.g., via use or inclusion of pigments, dyes or inks), are known in the art.

In an array of two or more packaged tampon products (herein “array” means two or more differing products of the same brand, marketed or appearing for sale simultaneously in the same or proximate respective locations (physical or online/virtual) (e.g., on the same or respective proximately located shelves within the same retail store)), differing secondary absorbents can be included with tampons of differing features, for the purposes of functioning differently with the differing tampon products, signaling the differences in features to consumers, or a combination of both. In one non-limiting example, packaged tampons having a first absorption capacity may include secondary absorbents imparted with a first color, while packaged tampons having a second, differing absorption capacity may include secondary absorbents imparted with a second color visually distinguishable from the first color. For purposes herein, a second secondary absorbent color is “visually distinguishable” from a first secondary absorbent color when the second secondary absorbent color exhibits a ΔE*≥5 from the first secondary absorbent color, measured according to the Color Measurement Method below. Secondary absorbents for an array of differing tampon products may differ not only in color, but in other characteristics such as material composition and/or absorbency/wicking characteristics, physical structure (e.g., braided, twisted, knitted, entangled, etc.), length, width, diameter, density, decitex or other dimension, location of attachment on the primary absorbent, etc. The differing tampon products with respectively differing secondary absorbents may be accompanied by associated packaging material imprinted with graphic/pictorial information, verbal information, or a combination thereof, that signals the differences in the respective products and/or secondary absorbents.

A tampon as contemplated herein is believed to offer several advantages over prior art tampons. As noted previously, the incorporation of the secondary absorbent 15 relatively longer structure extends fluid capturing capability to lower regions of the vaginal cavity. Additionally, because the tampon can be manufactured by processes in which the secondary absorbent is less compressed than the primary absorbent 11, the material forming the secondary absorbent can be available to immediately draw in fluid, without the need for re-expansion from a compressed state.

Tampons of the type and configuration(s) contemplated herein may be manufactured via the process described in U.S. Pat. No. 11,779,494 and/or U.S. Patent Publication No. 2020/0188190.

To form a tampon ready for use, the tampon primary absorbent 11 may be compressed and heat conditioned (which may include use of steam or elevated humidity) in any suitable conventional manner to impart it with a self-sustained form suitable for easy and comfortable insertion, which may be a cylindrical form. Pressures, temperatures and humidity conditions suitable for this purpose are known in the art. Typically, the primary absorbent 11 is compressed in both the radial and axial direction using any suitable means known in the art. While a variety of techniques are known and acceptable for these purposes, a modified tampon compressor machine available from Hauni Machines, Richmond, VA, is suitable.

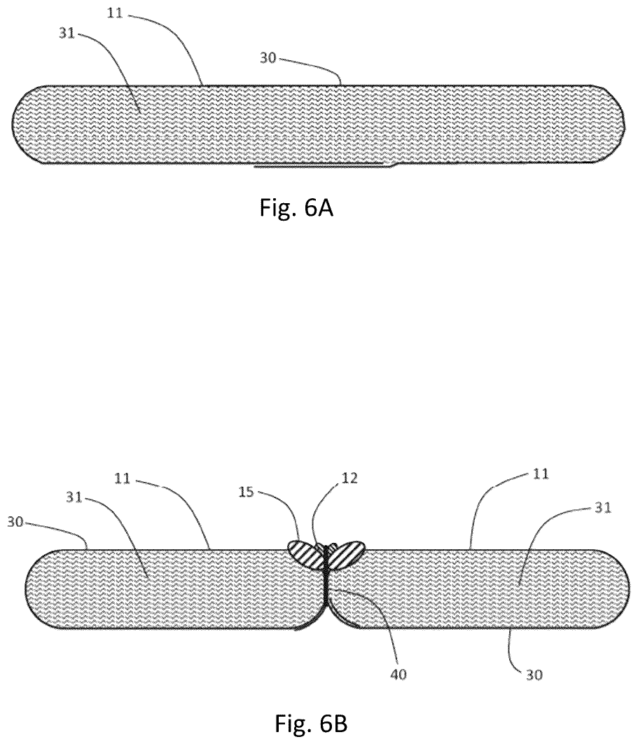

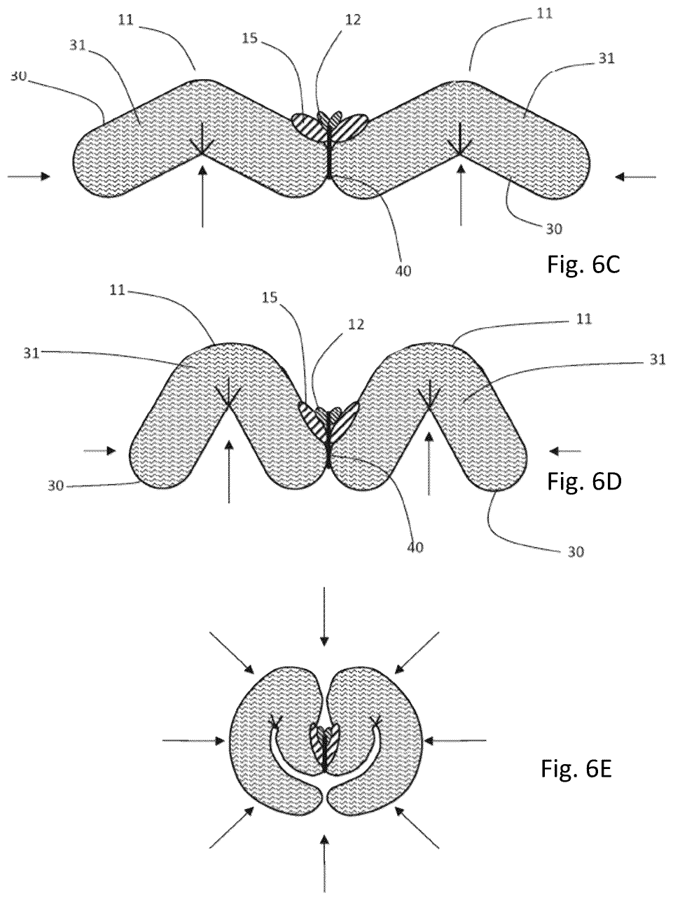

FIGS. 6A-6E sequentially illustrate in lateral cross section, the manner in which a non-limiting example of primary absorbent 11 having a configuration the same or similar to that described herein may have a secondary absorbent 15 and withdrawal member 12 joined thereto, such as by a line of stitching 40, and folded along longitudinal fold lines and subsequently molded under a combination of moisture control, applied heat and applied pressure and thereby compressed (as indicated by the arrows in FIG. 6E) to form it into a self-sustaining cylindrical or capsule-shaped form.

The tampon 10 contemplated herein may be inserted digitally or via the use of an applicator. If the tampon 10 is to be configured for digital insertion, or for insertion from a generally cylindrical applicator, it may be desirable to form the primary absorbent from a layer of absorbent material which has been rolled or otherwise formed into a cylindrical or capsule shape.

Any of the currently available tampon applicators may also be used for insertion of the tampon contemplated herein. Such applicators of typically a barrel and plunger type arrangement and may be plastic, paper, or other suitable material. A compact type applicator can also be suitable. The applicator plunger may be depressed by the user to push the compressed primary absorbent 11 out of the applicator while fitting around the secondary absorbent 15.

### Test Methods

### Off-Pad Secondary Absorbent Profile Method

The off pad secondary absorbent profile method measures the length and the segment mass of the portion of the secondary absorbent that extends beyond the base of the primary absorbent, also referred to herein as the pledget, on a tampon test sample. The secondary absorbent is excised from the primary absorbent and its length is measured. The excised secondary absorbent is then sliced into 10 mm long segments and the mass of each segment is measured. All measurements are performed in a laboratory maintained at 23° C.±2° C. and 50%±2% relative humidity.

Test samples are conditioned in a laboratory maintained at 23° C.±2° C. and 50%±2% relative humidity for at least 2 hours prior to testing. The tampon is removed from its wrapper and, from this point forward, it is carefully handled in such a way that prevents any tension or distortion to the secondary absorbent region and withdrawal member. A fresh, new razor blade (such as VWR Single Edge Industrial Razor blade No. 9, surgical carbon steel, or equivalent) is used to cut the applicator away from the tampon, using care to prevent any cuts to the underlying tampon. The compressed primary absorbent region of the tampon is opened and flattened, then placed onto a rigid horizontal work surface such that the side of the primary absorbent where the withdrawal member and secondary absorbent are attached faces the work surface. The secondary absorbent region that extends beyond the base of the primary absorbent and the withdrawal member are straightened without imparting any tension. The test specimen is prepared as follows. Another fresh, new razor blade is used to excise the secondary absorbent from the base of the primary absorbent by making a slice that is orthogonal to the length of the secondary absorbent at a location that is at the edge of the base of the primary absorbent. The primary absorbent is then discarded. In like fashion, a total of five replicate test specimens are prepared.

Measurements are made on the test specimen as follows. For the purposes herein, the length of the secondary absorbent along the withdrawal member of the prepared test specimen is defined as the entire region where any amount of the secondary absorbent material is present along the withdrawal member, and that region begins at the edge of the test specimen that was excised from the primary absorbent and continues until a point at which the presence of secondary absorbent material ends and only withdrawal member material remains. The length of the secondary absorbent is measured using a calibrated steel ruler (traceable to NIST or equivalent) and recorded as secondary absorbent length to the nearest 1 mm. The excised secondary absorbent is then sliced into five segments by making a series of slices that are orthogonal to the length of the secondary absorbent using another fresh, new razor blade, as follows. The first segment is a length of 0-10 mm from the primary absorbent, and it is prepared by making a slice at a location that is 10 mm from the end of the secondary absorbent that was excised from the base of the primary absorbent. The second segment is a length of 10-20 mm from the primary absorbent, and it is prepared by making a slice at a location that is 10 mm from the edge where the first segment was prepared. In like fashion, subsequent slices are made to prepare a third segment with a length of 20-30 mm from the primary absorbent, a fourth segment with a length of 30-40 mm from the primary absorbent, and a fifth segment with a length of 40-50 mm from the primary absorbent. The mass of each segment is measured and recorded as total segment mass to the nearest 0.1 mg along with the corresponding segment label (i.e., first, second, third, etc.). A sub-specimen of the withdrawal member is prepared by slicing the cord to create a 50 mm long segment that contains only withdrawal member material. The mass of the 50 mm long withdrawal member is measured and recorded as total withdrawal member segment mass to the nearest 0.1 mg. The total withdrawal member segment mass is divided by the length of the withdrawal member segment (50 mm) then multiplied by 10 mm (the length of each segment of secondary absorbent material) and recorded as withdrawal member mass per secondary absorbent segment to the nearest 0.1 mg. Now subtract the withdrawal member mass per secondary absorbent segment from the total segment mass for each of the five segments of the secondary absorbent, and record as secondary absorbent segment mass to the nearest 0.1 mg along with the corresponding segment label (i.e., first, second, third, etc.). In like fashion, the entire procedure is repeated for all five replicate test specimens.

The arithmetic mean of the secondary absorbent length is calculated among the five replicate test specimens and reported as the mean length from the primary absorbent to the nearest 1 mm. The arithmetic mean of the secondary absorbent segment mass for the first segment is calculated among the five replicate test specimens, and reported as the mean segment mass, at a length of 0-10 mm, to the nearest 0.1 mg. In like fashion, the arithmetic mean of the secondary absorbent segment mass is calculated for the remaining segments (second, third, fourth, and fifth) and reported, respectively, as mean segment mass at a length of 10-20 mm, mean segment mass at a length of 20-30 mm, mean segment mass at a length of 30-40 mm, and mean segment mass at a length of 40-50 mm, all to the nearest 0.1 mg.

The standard deviation (SD) of the secondary absorbent mean segment mass for each of the segments is calculated using the following equation:

$$SD = \sqrt{\frac{\sum {\vert x - μ \vert}^{2}}{N}}$$

where Σ means “sum of”, x is an individual value of the secondary absorbent segment mass in the data set of the given segment, μ is the mean segment mass of the given segment, and N is the number of data points in the population of the given segment (5). The calculated standard deviation of the secondary absorbent mean segment mass for each segment (first, second, third, fourth, and fifth) is reported, respectively, as SD of the mean segment mass at a length of 0-10 mm, SD of the mean segment mass at a length of 10-20 mm, SD of the mean segment mass at a length of 20-30 mm, SD of the mean segment mass at a length of 30-40 mm, and SD of the mean segment mass at a length of 40-50 mm, all to the nearest 0.1 mg.

### Tampon Pusher Drag Force Method

The drag force that results between contact of a test specimen that includes the withdrawal member and secondary absorbent region of a tampon and the inner walls of the pusher component of the tampon applicator is measured as the entire length of the test specimen is pulled through the entire length of the pusher. Drag force is measured using a universal constant rate of extension test frame fitted with a custom fixture. All measurements are performed in a laboratory maintained at 23° C.±2° C. and 50%±2% relative humidity and test specimens are conditioned in this environment for at least 2 hours prior to testing.

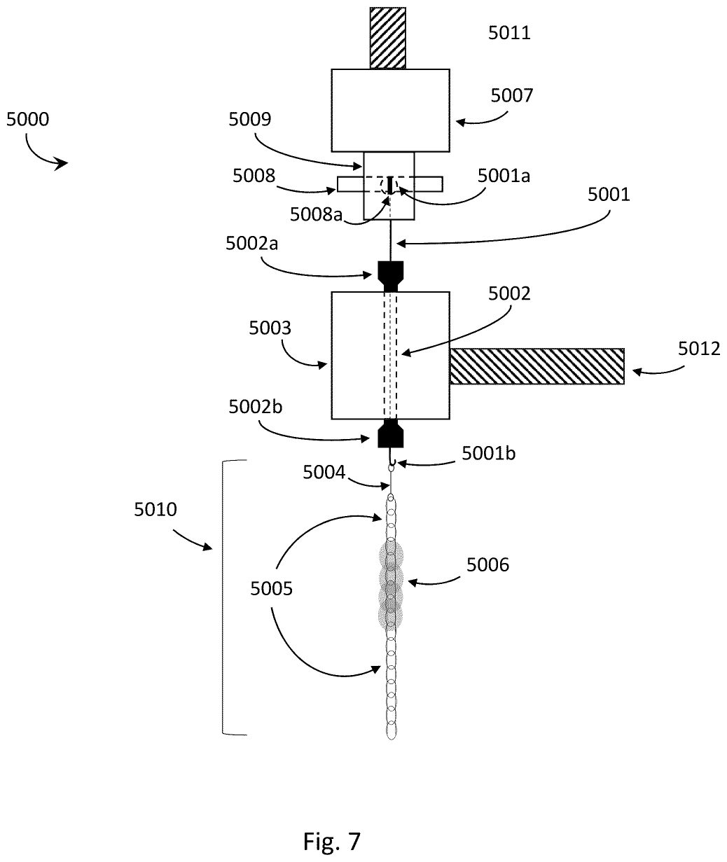

The test setup is depicted in FIG. 7 and consists of a universal constant rate of extension test frame fitted with a custom fixture assembly 5000 that provides a means to hold the pusher component of the tampon applicator stationary while the entire length of the test specimen is pulled through the entire length of the pusher. A suitable universal constant rate of extension test frame is the MTS Alliance interfaced to a computer running TestSuite control software (available from MTS Systems Corp, Eden Prairie, MN), or equivalent. The universal test frame is equipped with a load cell 5007 for which forces measured are within 1% to 99% of the limit of the cell. The load cell 5007 is mounted to the upper moveable crosshead 5011 of the test frame, and a pin holder 5009 is then mounted to the load cell 5007. The pin 5008 is made of stainless steel with a length of about 44 mm and a diameter of 6.3 mm. The pin 5008 is modified such that a groove 5008a is milled around its circumference at its longitudinal midpoint such that when the wire extender 5001 is mounted on the pin 5008, the groove 5008a holds the extender 5001 centered along the motion of the crosshead and prevents lateral movement of the extender 5001 during the test. The groove 5008a is milled to a depth of about 0.8 mm. The wire extender 5001 is composed of heavy gauge stainless steel wire that has a diameter of about 1 mm. One end of the extender 5001 is formed into a loop 5001a that has an outer diameter of about 9 mm. The other end of the extender 5001 is formed into a hook 5001b that has a length of about 4 mm and an outer width of about 2.5 mm. With the ends formed into the loop 5001a and hook 5001b, the extender 5001 has an overall length of about 135 mm. The loop 5001a of the extender 5001 is inserted into the pin holder 5009, then the pin 5008 is inserted orthogonally through the pin holder 5009 such that it passes through the loop 5001a of the extender 5001. The loop 5001a of the extender 5001 is positioned into the groove 5008a of the pin 5008, and the position of the pin 5008 is adjusted such that the extender 5001 is parallel and centered with the motion of the crosshead. A custom pusher holder 5003 is mounted to the lower stationary crosshead 5012 of the test frame such that the pusher 5002 is held with its longitudinal axis parallel and centered with the motion of the crosshead. The overall dimensions of the pusher holder 5003 are unique to the size and shape of the pusher 5002 being tested, and it consists of a removable face that allows the pusher 5002 to be mounted within. The holder 5003 must be rigid and nondeformable, and the interior cavity must conform to the shape of the outside walls of the pusher 5002 being tested such that when the pusher 5002 is mounted within the holder 5003, it is held stationary in a manner that prevents any deformation or collapse of the walls of the pusher 5002. The pusher holder 5003 is designed such that it holds the long axis of the cylindrical portion of the pusher 5002, excluding any flanged or otherwise non-cylindrically shaped portion that may be present at the ends (5002a and 5002b) of the pusher 5002. One suitable way to fabricate the custom pusher holder 5003 is to create a 3D-printed plastic mold. This type of design ensures the pusher 5002 component of the tampon applicator remains in a fixed position as the test specimen 5010 is pulled through its interior. It is understood that different samples will have a uniquely shaped applicator pusher, and thus a new pusher holder 5003 will need to be fabricated for each.

Tampon test samples are conditioned in a laboratory maintained at 23° C.±2° C. and 50%±2% relative humidity for at least 2 hours prior to testing. The tampon is removed from its wrapper and, from this point forward, it is carefully handled in such a way that prevents any tension or distortion to the withdrawal member and secondary absorbent region. A fresh, new razor blade (such as VWR Single Edge Industrial Razor blade No. 9, surgical carbon steel, or equivalent) is used to cut the barrel of the applicator away from the tampon and the pusher component of the applicator, using care to prevent any cuts to the underlying tampon and pusher. The cut-open barrel of the applicator is discarded and the pusher component is retained for the test. The compressed primary absorbent of the tampon is opened and flattened, then placed onto a rigid horizontal work surface such that the side of the primary absorbent where the withdrawal member and secondary absorbent are attached faces the analyst. The test specimen is prepared as follows. Another fresh, new razor blade is used to excise the withdrawal member, including the secondary absorbent, from the primary absorbent by carefully slicing the material, such as thread, that attaches the withdrawal member and secondary absorbent to the primary absorbent in such a way that prevents any cuts to the withdrawal member and secondary absorbent. After the test specimen that includes the secondary absorbent 5006 and withdrawal member 5005 has been excised from the primary absorbent, the primary absorbent is discarded. The test specimen 5010 is further prepared for testing as follows. A piece of extra strong, non-extensible nylon sewing thread (any convenient source) is cut to a length of about 140 mm. One end of the thread is tied off to form a loop that has a diameter of about 15 mm. The other end of the thread is securely tied to the test specimen at the end of the withdrawal member that was previously joined to the primary absorbent. Once attached to the test specimen, the thread 5004 has an overall extended length of about 90 mm. In like fashion, a total of three replicate test specimens are prepared.

Referring to FIG. 7, the test frame and test specimen 5010 are prepared for the drag force test as follows. The retained pusher 5002 component of the tampon applicator is secured into the custom pusher holder 5003. The pusher holder 5003 is then mounted to the stationary crosshead 5012 of the test frame such that the pusher 5002 is oriented with is longitudinal axis parallel and centered with the motion of the crosshead, and the barrel end of the pusher 5002a, which was disposed within the barrel in the assembled tampon, is facing the upper, moveable crosshead 5011 of the test frame and the finger end of the pusher 5002b, which was the end that was external to the barrel in the assembled tampon, is facing the lower, stationary crosshead 5012 of the test frame. The modified pin 5008 and double hook extender 5001 are mounted onto the moveable crosshead 5011 as previously described. With the hook end 5001b of the extender 5001 aligned with the opening of the barrel end 5002a of the pusher 5002, the moveable crosshead 5011 is lowered such that the extender travels through the inside of the pusher 5002 until about 5 mm of the hook end 5001b of the extender 5001 is exposed at the finger end 5002b of the pusher 5002. When the extender 5001 and pusher 5002 are mounted and properly aligned, the extender 5001 will make no contact with the inside walls of the pusher 5002 as it travels through the interior of the pusher 5002. The crosshead is then zeroed. The prepared test specimen 5010 is now attached to the hook end 5001b of the extender 5001 by placing the loop end of the thread 5004 onto the hook. In this manner, the test specimen 5010 hangs freely from the hook end 5001b of the extender. The load cell 5007 is then zeroed. The test frame is programmed to pull the entire length of the test specimen 5010 (withdrawal member portion 5005 and the secondary absorbent portion 5006) through the entire length of the pusher 5002 at a rate of 5 mm/s while force (gram-force) and extension (mm) are continuously recorded. A graph depicting load (gram force) versus extension (mm) is constructed. The maximum load value is recorded as peak force to the nearest 0.01 grams. In like fashion, the procedure is repeated until all three replicate test specimens have been analyzed.

The arithmetic mean of the peak force values recorded among the three replicate test specimens is calculated and reported as pusher peak drag force to the nearest 0.01 grams.

### Color Measurement

The total color difference (ΔE*) between a tampon primary absorbent 11 and its secondary absorbent 15 is calculated from the L* a* b* color values obtained for each respective portion of the tampon. Color analyses are made using a 0°/45° spectrophotometer with adjustable apertures capable of making standard CIE L*a*b* measurements in accordance with ASTM E1349. An example of a suitable spectrophotometer is the Labscan XE (available from Hunter Associates Laboratory, Inc., Reston, VA, or equivalent). All testing is performed in a room maintained at a temperature of 23° C.±2.0° C. and a relative humidity of 50%±2% and samples are conditioned under the same environmental conditions for at least 2 hours prior to testing.

If the tampon is provided in an application, the test sample is prepared by first removing the tampon 10 from the applicator in the manner the product is designed to effect ejection of the tampon 10 from the applicator. The primary absorbent 11 is flattened out by gently opening it from its self-sustaining shape. Using a small pair of scissors to sever any stitching as necessary, or using freeze spray to deactivate any adhesive used to join them, gently separate and remove the secondary absorbent 15 from the primary absorbent 11, using care so as not to damage either component in the process. For each product tested, a total of 5 primary absorbents and 5 secondary absorbents are prepared in this manner.

To measure color, calibrate and standardize the instrument per the vendor instructions using the standard white and black tiles provided by the vendor. Set the spectrophotometer to use the CIE L*a*b* color space with a D65 standard illumination, a 10° observer, a 0.125 inch area view, a 0.200 inch aperture, and the UV filter set to nominal. Place the pre-flattened primary absorbent test sample over the aperture such that the entire aperture is covered by the primary absorbent 11 on an area free of the withdrawal string 40. Place the standard white tile behind the pre-flattened primary absorbent test sample, take a reading and record L*a*b* values as L₂*a₂*b₂* to the nearest 0.01 units. Remove the primary absorbent test sample from the aperture and replace it with the secondary absorbent test sample. Ensure that the entire aperture is covered by the secondary absorbent, minimizing the amount of withdrawal member 12 present in the aperture's viewing area. Place the standard white tile behind the secondary absorbent test sample, take a reading and record L*a*b* values as L₁* a₁*b₁* to the nearest 0.01 units. Calculate the total color difference (ΔE*) between the primary absorbent and the secondary absorbent as follows:

$${{{{Δ E *= [ ( {L}_{2} * - {L}_{1} \text{*)}}^{2} + ( {a}_{2} * - {a}_{1} \text{*)}}^{2} + ( {b}_{2} * - {b}_{1} \text{*)}}^{2} ]}^{1 / 2} ,$$

and record as ΔE* to the nearest 0.01 units.

In like fashion, repeat for a total of five measurements obtained on five different tampon primary absorbent and secondary absorbent samples. Calculate the arithmetic mean for ΔE* obtained from all five measurements and report to the nearest 0.01 unit.

### Wicking Measurement

The ability of a tampon configuration with a secondary absorbent to capture fluid in the secondary absorbent, and wick and fluid to the primary absorbent, may be measured using this Wicking Measurement method. A known quantity of test fluid is delivered at a constant rate over a specified amount of time to a portion of the secondary absorbent 15 inside a pressurized wicking chamber. The quantity of fluid absorbed by the tampon 10 is determined and reported as Total Uptake. All measurement is performed in a laboratory maintained at 23° C.±2° C. and 50%±2% relative humidity. The measurement equipment as described herein is configured to approximate the pressure to which a tampon is subjected inside the body during actual use.

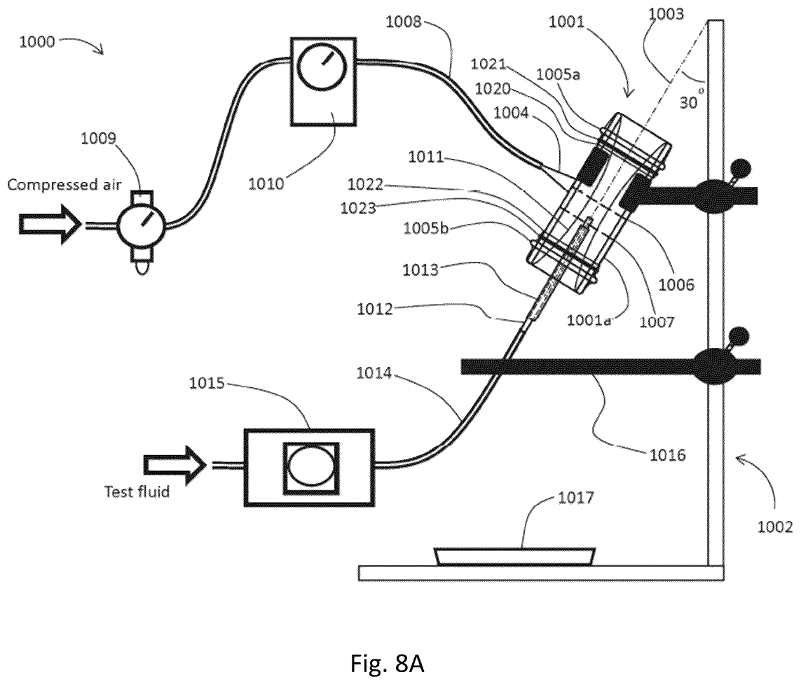

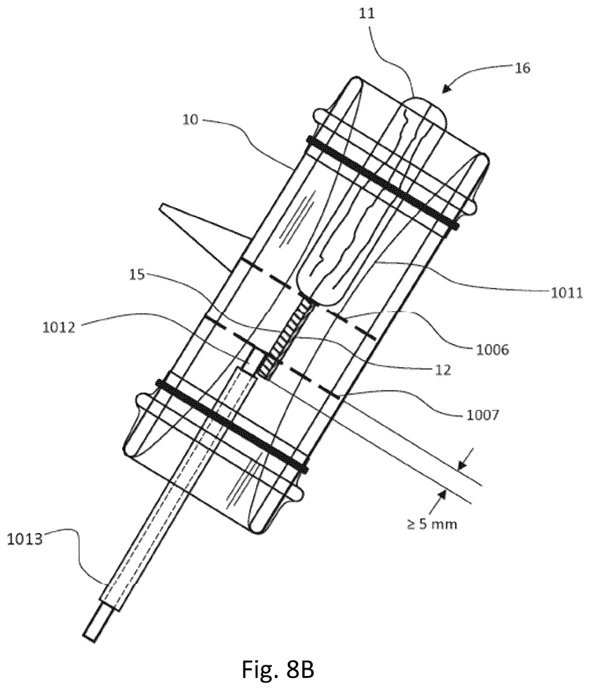

The measurement apparatus 1000 is schematically depicted in FIG. 8A, with additional details illustrated in FIG. 8B. The measurement apparatus 1000 includes a wicking chamber 1001 including a hollow glass tube 1001a mounted to a ring stand 1002 in such a way that the longitudinal axis 1003 of the tube 1001a lies within a vertical plane and is 30°+2° from a vertical line. The glass tube 1001a is open at both ends, and has a 3.3 cm inner diameter, a 3.8 cm outer diameter, and an overall length of 10.5 cm. The glass tube 1001a has a ¼ inch hose barb 1004 (that provides fluid communication with the space inside the tube) located at its longitudinal midpoint, extending perpendicularly from the outer surface of the glass tube. Located about 7 mm from each end of the glass tube are circumferential sealing ridges 1005a, 1005b on the outside of the tube (which may be formed as “marias,” also known as “mariahs,” as known in glassblowing art), each with a 48.0 mm outer diameter and oriented along a plane perpendicular to the longitudinal axis 1003 of the glass tube. Two lines 1006, 1007 are marked on the glass tube 1001a, both oriented along planes perpendicular to the longitudinal axis 1003 of the tube: the Pledget Base Line 1006 is marked at the longitudinal midpoint of the glass tube; the Cannula Position Line 1007 is marked 10.0 mm below the Pledget Base Line 1006. A compressed air source is connected to the hose barb 1004 with flexible tubing 1008. The air pressure is controlled via a pressure regulator 1009 and calibrated manometer 1010 (standardized to ANSI standards). The pressure regulator 1009 is set to 0.5±0.02 psi.

An unlubricated condom 1011 (condom complying with ASTM D3492) is installed inside the glass tube 1001a as follows. Unroll the condom 1011 and mark a Positioning Line thereon that is perpendicular to the longitudinal axis of the condom 1011 and located 12.0±0.1 cm from the open end.

Using a glass rod inserted into the condom 1011, push the condom 1011, closed/tip end first, into the lower open end of the glass tube 1001a, up through the tube and out the upper open end. Cut off the tip of the closed end of the condom 1011 (no more than about 1 cm from the tip) and discard. Align the Positioning Line marked on the condom with the edge of the open upper end of the glass tube 1001a. Now, stretch and pull the newly cut edge 1020 of the condom 1011 radially out, down and over the circumference of the upper edge of the glass tube 1001a, and downwardly over and past the upper sealing ridge 1005a. Secure the circumferential cut edge of the condom 1011 to the outside of the glass tube 1001a below the upper sealing ridge 1005a, using a first rubber band 1021. Now, from the bottom, gently pull the condom slightly taut longitudinally, and stretch the circumferential edge 1022 of the original open end of the condom 1011 radially out, up and over the circumference of the lower edge of the glass tube 1001a and upwardly past the lower sealing ridge 1005b, and secure the circumferential edge of the original open end of the condom 1011 to the outside of the glass tube 1001a above the lower sealing ridge 1005b, using a second rubber band 1023.

(The references “up”, “down”, “upper”, “lower”, and similar terms used in this measurement method description are relative the position of the glass tube when mounted to and held by the ring stand as shown in FIG. 8A. However, the condom may be installed within the glass tube before it is mounted on the ring stand. Similarly, the Pledget Base Line and Cannula Position Line on the glass tube as described above may be marked on the tube before it is mounted on the ring stand.)

The test fluid used (for purposes of providing a fluid having a suitable degree of similarity to human menstrual fluid) is defibrinated sheep's blood, with a packed cell volume between 38%-42% (such as that available from Cleveland Scientific Ltd., Bath, OH, or equivalent) and a viscosity between 6.5-8.0 centistokes. Prior to use in this measurement method, the viscosity of the test fluid is measured using a low viscosity rotary viscometer (a suitable instrument is the Cannon LV-2020 Rotary Viscometer with UL adapter, Cannon Instrument Co., State College, PA, or equivalent). The appropriate size spindle for the viscosity range is selected, and the instrument is operated and calibrated as per the manufacturer's instructions. Measurements are taken at 23° C.±1° C. and at 60 rpm. Results are recorded to the nearest 0.01 centistokes and must be in spec before use.

The test fluid is placed in a 250 mL reservoir with a cover and continuously and moderately stirred to avoid separation. The temperature of the test fluid is maintained at 23° C.±2° C. during use. The test fluid is supplied from the reservoir to a 15-gauge steel laboratory cannula 1012 (about 10 cm long with a blunt tip at each end with an inner diameter of 1.33-1.41 mm and an outer diameter of 1.82-1.84 mm (such as that available from Cadence Science Inc., 2080 Plainfield Pike, Cranston, RI 02921, or equivalent) and cannula sleeve 1013 (transparent flexible silicone tubing with 3.2 mm inner diameter, 4.8 mm outer diameter, about 7 cm long (such as that available from Cole Parmer, Verner Hills, IL, or equivalent) with peristaltic pump tubing 1014 that has an inner diameter of 1.6 mm. The cannula 1012 is inserted through the prepared cannula sleeve 1013 such that there is about 6 mm of the upper/distal tip of the cannula 1012 extending beyond the upper/distal edge of cannula sleeve 1013. (The purpose of the cannula sleeve, of an inner diameter greater than the outer diameter of the cannula, is to provide a pathway for fluid that is not captured and wicked after contacting the secondary absorbent, to flow under gravitational pull down and out of the test chamber, thereby helping reduce chances of pooling of the fluid within the test chamber.) The lower end of the cannula 1012 is inserted into the peristaltic pump tubing 1014. A peristaltic pump 1015 (such as Master Flex, available from Cole Parmer, Verner Hills, IL, or equivalent) is programmed to deliver 5.0 g±0.25 g of test fluid at 1.0 g/min±0.02 g/min (i.e., over a 5-minute period of operation of the pump). Prior to commencement of the measurement, the peristaltic pump 1015 is calibrated with the test fluid, and the tubing 1014 and cannula 1012 are then primed with test fluid.

A cannula stabilizing bar 1016 is mounted to the ring stand 1002 such that it is about 2 cm below the bottom of the wicking chamber 1001, however, this position can be adjusted as needed. The cannula stabilizing bar 1016 is used to support the peristaltic pump tubing 1014 in order to allow the cannula 1012 to be maintained in a position in which its longitudinal axis is approximately parallel to longitudinal axis 1003 of the glass tube 1001a during the measurement.

Tampon measurement samples still in their applicators and wrappers are conditioned at 23° C.±2° C. and 50±2% relative humidity for at least 2 hours prior to use. Measurement samples are not removed from their wrappers or applicators until immediately prior to use, and must be used within 30 minutes following removal. Clean disposable exam grade, nitrile rubber, powder-free medical gloves must be worn while preparing the measurement samples and during the measurement procedure in order to prevent any contamination from contact with the analyst's hands. Each tampon measurement sample is prepared by first removing the tampon 10 from its applicator in the manner the product is designed to effect ejection of the tampon from the applicator. Cut the withdrawal member 12 from the tampon 10 at the rear end of the trailing portion 15b of the secondary absorbent 15. After cutting away such portion of the withdrawal member, record the Dry Mass of the tampon measurement sample to the nearest 0.01 g.

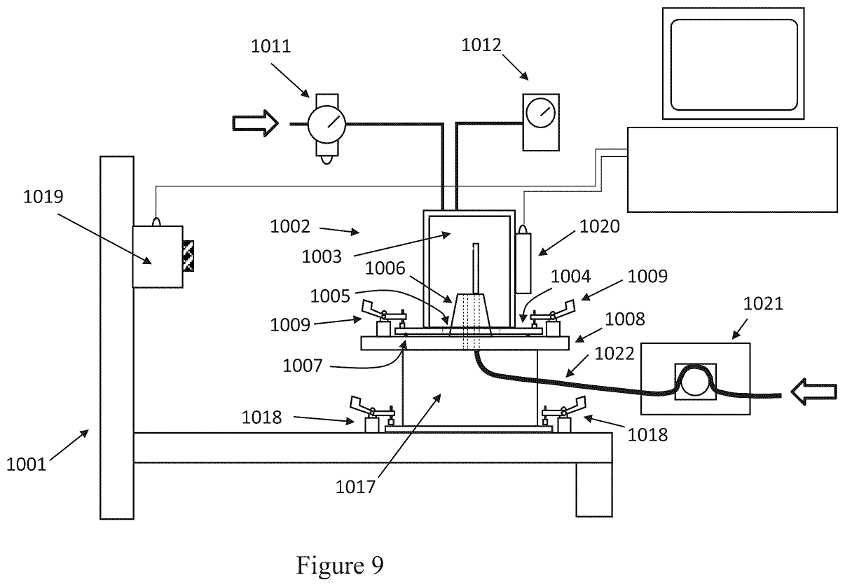

Now referring to FIG. 9B, the tampon measurement sample is placed into the unpressurized wicking chamber 1001 as follows. (A second analyst may be required to assist in manipulating the measurement sample and cannula into position and holding them in position until the measurement chamber is pressurized.) Insert the forward end 16 of the primary absorbent 11 into the bottom of the wicking chamber 1001 and move the sample upward in the chamber to a location at which the rearward end 17 of the primary absorbent 11 is aligned with the Pledget Base Line 1006. Align the longitudinal axis of the primary absorbent and secondary absorbent 15 approximately with the longitudinal axis 1003 of the wicking chamber 1001. If the secondary absorbent 15 and remaining portion of withdrawal member 12 are not coaxial, rotate the tampon measurement sample about its longitudinal axis within the chamber, to a position in which the trailing portion 15b of the secondary absorbent 15 occupies a predominately dorsal (overlying) position relative the remaining portion of the withdrawal member, and the remaining portion of the withdrawal member 12 occupies a predominately ventral (underlying) position relative the trailing portion 15b of the secondary absorbent. While holding the measurement sample in place inside the wicking chamber 1001 in this position, insert the prepared cannula 1012 with attached cannula sleeve 1013 into the bottom of the wicking chamber 1001 and move it upward within the chamber to a position at which the upward tip of the cannula 1012 is aligned with the Cannula Position Line 1007, and positioned directly over and in contact with the dorsal surface of the secondary absorbent 15, and overlaps it by at least 5 mm. (If the trailing portion 15b of secondary absorbent 15 of the particular tampon sample is shorter than 15 mm when in a straightened but substantially relaxed condition so as to provide ≥5 mm overlap, position the cannula tip over the secondary absorbent so that it overlaps the trailing end of the secondary absorbent by ⅓ of the length of the trailing portion 15b. If the trailing portion 15b of secondary absorbent 15 of the particular tampon sample is shorter than 6 mm when in a straightened but substantially relaxed condition, then the sample cannot be tested according to this method, and does not fall within the contemplation of claims herein that recite a value for Wicking.)

Now, pressurize the wicking chamber 1001 to 0.5±0.02 psi (over ambient air pressure) such that the condom 1011 inflates within the wicking chamber around the measurement sample and cannula 1012 to hold them in place inside the chamber 1001. Ensure that there is still a distance of 6 mm between the tip of the cannula 1012 and the edge of the cannula sleeve 1013. Adjust the position of the cannula stabilizing bar 1016 so that it supports the peristaltic pump tubing 1014 to allow the longitudinal axis of the cannula 1012 to be maintained in a position that is approximately parallel to the longitudinal axis 1003 of the wicking chamber 1001. Adjust as needed to maintain this position throughout the measurement.

Prior to starting of the peristaltic pump, a tray 1017 or other suitable collection means may be placed on the benchtop below the bottom of the cannula sleeve 1013 and wicking chamber 1001 to collect any test fluid that is not wicked/absorbed by the measurement sample and exits the bottom of the test chamber.

Start the peristaltic pump 1015 to deliver 5.0 g±0.25 g of test fluid at 1.0 g/min±0.02 g/min through the cannula 1012. While the test fluid is being delivered, ensure that the tip of the cannula 1012 remains in contact with the trailing portion 15b of the secondary absorbent 15 and that there is no pooling of fluid in a crease in the condom 1011 proximate the cannula tip. If pooling of test fluid is observed, move the cannula 1012 slightly further down the trailing portion 15b of the secondary absorbent 15. If pooling of test fluid continues, discard the tampon and repeat using a new measurement sample.

After the pump stops, depressurize the wicking chamber 1001 and remove the test sample. Record the Wet Mass of the test sample to the nearest 0.01 g. Calculate the mass of fluid absorbed by the test sample as Wet Mass-Dry Mass and record as Total Uptake to the nearest 0.01 g. The condom 1011 is wiped clean in between test samples and replaced after every 10 samples are tested.

In like fashion, repeat for a total of ten replicate test samples. Calculate the arithmetic mean for Total Uptake measured across all ten replicate test samples and report to the nearest 0.01 g.

### Tampon Expulsion Force

The expulsion force can be measured by using a Tampon Expulsion Force Test. The Tampon Expulsion Force Test determines the force characteristics of tampon expulsion from an applicator. The test can utilize a constant rate of elongation tensile tester with a load cell chosen such that the measure force is between 10% and 90% of the cell's capacity. The test can also utilize an applicator fixture that must be able to hold applicator vertically downward (petals facing down) while exerting minimal force to the axial portion of the applicator.

The tester and fixture is placed in a room with a temperature of 21° C. (±4° C.). This uniform temperature allows for comparison between products that expand based on temperature.

### Sample Preparation (if Needed):

A test sample is prepared by removing any outer wrapper that may exist and tucking any out-hanging withdrawal cord inside the applicator pusher.

The applicator is placed into fixture such that the lip of the applicator is being held up (opposite to the direction of testing, preventing applicator from falling through the fixture). The fixture is then oriented within the tensile tester setup such that the direction of the force of the tensile tester should follow the long direction of the applicator and be applied directly on the pusher (plunger). The test is then run (speed: 1 mm/sec, data acquisition 20 hz) where the tensile tester is exerting downward force directly on the pusher such that the tampon moves toward and out of the petals. Test is complete when the body of the tampon has been completely expelled from the applicator.

Reporting of the results includes obtaining the force vs extension curves and determining the maximum expulsion force, as well as the number of peaks in the curve, at what displacements they occurred and the corresponding loads.

### Tampon Density

The following steps are followed to calculate the density of a tampon according to the present invention. The tampon withdrawal string (or comparable withdrawal structure) and any existing secondary absorbent feature (e.g., a braid) is cut at the bottom of the tampon absorbent body. The tampon body is then weighed to the nearest 0.01 grams. And the tampon body length is measured to the nearest 0.1 millimeters. The tampon body is then immerse into isopropyl alcohol for 30 seconds or until air bubbles stop. Remove the tampon from the alcohol and allow the excess alcohol to drip from the tampon for 15 seconds. Separately a test cylinder with spout is filled with isopropyl alcohol until it overflows at the spout into a 50 milliliter beaker. Tare the 50 milliliter beaker. Place the tampon body after the excess has dripped from the same slowly into the cylinder containing the standard volume of isopropyl alcohol. Collect the overflow of isopropyl alcohol from the spout that resulted from the addition of the tampon body. Obtain the weight of the isopropyl alcohol that overflowed the test cylinder to the nearest 0.01 grams.

$$Tampon volume ( milliliters ) = weight of overflowed alcohol / 0.78 grams per milliliter$$

$$Tampon density ( grams per milliliter ) = tampon weight ( grams ) / tampon volume$$

### Dynamic Expansion

Expansion Width is measured from the change in diameter of a tampon under pressure as test fluid is introduced at a specified flow rate. The expansion at three sites along the length of the tampon is measured at a specified fluid capacity and averaged to give the Expansion Width to the nearest 0.01 mm.

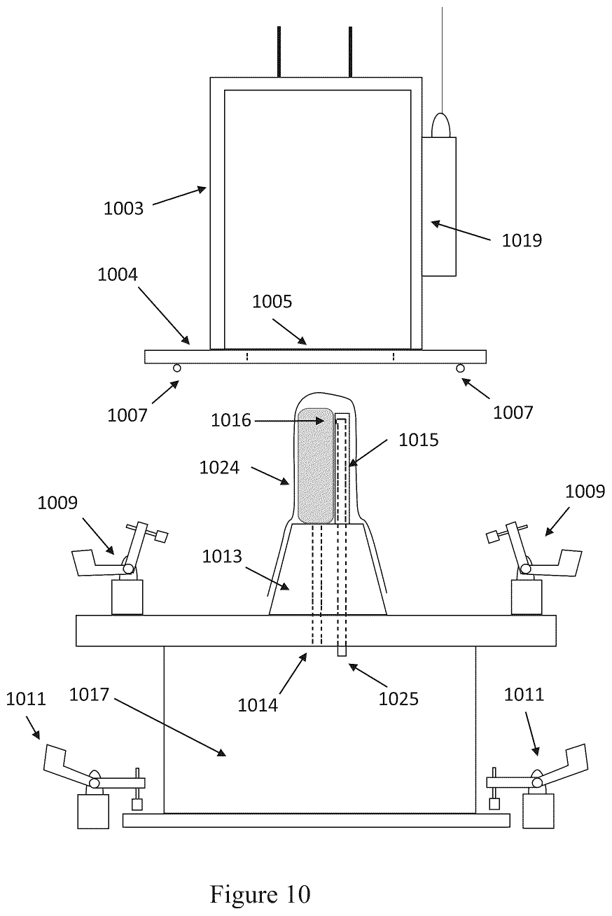

Referring to FIG. 9 and FIG. 10, the test apparatus includes a frame 1001 that supports the expansion assembly 1002. The assembly is constructed of a pressurized Plexiglas chamber 1003 with a bottom plate 1004. The plate has a circular opening 1005 such that the tampon mounting stand 1006 can be inserted through the opening. The bottom plate also has a silicone gasket 1007 that surrounds the opening 1005 and is used to seal the chamber 1003 against the mounting platform 1008 using clamps 1009. The chamber is pressurized to 3.45 kPa using a compressed air source 1010. The chamber pressure is adjusted via a pressure regulator 1011 and calibrated manometer 1012 (standardized to ANSI standards).

The tampon mounting stand 1006 is fixed to the mount plate 1008 and is made up of a tapered cylindrical base 1013 with a 3 mm diameter drain hole 1014 that extends from the top of the base and through the mount plate. A Plexiglas delivery tube 1015 (9.5 mm O.D by 3 mm I.D.) extends 48.5 mm above base 1013. The fluid path originates from a tube connector 1025 below the mount plate 1008 and extends through the mount base and terminates in a 1.5 mm opening 1016 on the side of the delivery tube 1015 (the top of the tube is sealed). The opening is 5 mm down from the top of the tube and faces the tampon. The mounting platform 1008 has a base 1017 that is open on two of its sides (to allow cleaning of fluid that drains from the drain hole 1014) and clamps 1018 to the frame 1001.

The imaging system consist of a sensor/camera 1019 (gray scale, minimum of 640×480 pixels, such as the Cognex DVT 545, available from PDF Supply, Cary NC, or equivalent) and is attached to the frame 1001 and light box 1020 (such as the Cognex DVT Smart Light, also available from PDF Supply, Cary NC, or equivalent) for backlighting is attached to the pressure chamber 1003. The camera is adjusted such that the total length of the sample is contained in the field of view. The system collects images and adjusts the threshold such that the sample in the foreground is dark against a light background. The digital images are analyzed using software suitable for making calibrated linear measurements. The software is calibrated for linear dimensions to the nearest 0.01 mm using standard cylinders of known diameters ranging from 12 to 24 mm. The horizontal width of the sample is measured at three sites; the “top” at 28% of the tampon length, “middle” at 50% of the tampon length, and “bottom” at 67% of the tampon length. All positions measured from the top of the tampon.

The test fluid used is Defibrinated Sheep's Blood, minimum 38% packed cell volume (available at Cleveland Scientific Ltd., Bath, OH, or equivalent). The test fluid is placed in a 250 mL reservoir and continuously and moderately stirred to avoid separation. The fluid is maintained at 23° C.±3° C. during use. The test fluid is supplied from the reservoir to the delivery tube 1015 with peristaltic pump tubing 2023. A peristaltic pump 1021 (such as Master Flex, available from Cole Parmer, Verner Hills, IL, or equivalent) delivers the test fluid at 1.0 g/min±0.02 g/min. The peristaltic pump tubing and delivery tube are filled before the start of testing.

Samples still in their applicators and wrappers are conditioned 23° C.±3° C. and 50±5% relative humidity for 4 hours prior to testing. Samples are not removed from their wrappers or applicators until immediately prior to testing. Remove the tampon from its applicator and cut the withdrawal string at the base of the tampon. If a braid, also referred to as a secondary absorbent, or skirt is present on the tampon, it is left intact. Measure the dry mass of the tampon to the nearest 0.01 gram and report as the Dry Tampon Mass (g). Using a digital Vernier caliper (e.g. Digimatic 500 series, Mitutoyo, or equivalent) measure the maximum length (not including skirt or braid if present) and maximum width and record each to the nearest 0.01 mm. This width is the Tampon Dry Width.

Place a small piece of 2-sided tape onto the sample side of the delivery tube 1015 making sure not to cover the side opening 1016. If the tampon's construction is a compressed, flat, sewn pad, mount the tampon so that the sewn side seam is toward the fluid delivery tube. For other constructions, direct any seam, if present, toward the delivery tube. Mount the sample onto the delivery tube with the top of the sample extending 2 mm above the top of the tube. If the sample length is greater than the height of the delivery tube, align the bottom of the sample to the top of the mounting base 1006. If the sample contains a braid or skirt, allow that structure to drape down the mounting base (opposite the delivery tube), assuring that it does not protrude past the width of the sample.

Unroll an unlubricated condom 2024 (condom compiling to ASTM 3492) and place loosely over the sample, delivery tube and upper portion of mounting base 1006 such that the end extends approximately 2 cm above the top of the sample. Place the expansion assembly over the sample and mounting base and clamp in place. Turn on the compressed air and adjust the pressure in the assembly to 3.45 kPa as read by the manometer. The condom should now be snug against the tampon. Start the vision system and inspect the resulting image. If folds of the condom appear at the sides or on top of sample in image, open chamber, readjust the condom, and repeat this step until the condom is smooth along the sides of the tampon in the image, e.g., no creases/wrinkles are visible.

Program the imaging system to take an image every 5 sec. Start the program and peristaltic pump. Continue flow to the sample until test fluid is detected at the bottom of the drain tube 1014. With the calibrated image software measure the width of the at the “top”, “middle” and “bottom” sites for all images taken and record to the nearest 0.01 mm. Calculate the Corrected Widths as:

$$Corrected {Width}_{[ s , i ]} = Dry Tampon Width + {Width}_{( s , i )} - {Width}_{[ s , 0 ]}$$

Where s=site (Top, Middle, or Bottom); i=time (in sec); all widths in mm

From these values, separate curves for the Corrected Width (mm) vs Time (sec) can be constructed for each measured site.

Expansion Width is defined as the Corrected Width at a size-specific test mass. For each tampon select the Test Time for the appropriate absorbency term from Table 3. From each of the site Corrected Width vs Time curves read and record the Corrected Width for the “Top”, “Middle” and “Bottom” sites at that selected Test Time. Calculate the arithmetic average of the three values and report as the Expansion Width to the nearest 0.01 mm.

In like fashion analyze 6 replicate samples, calculate the arithmetic average and report as the Expansion Width to the nearest 0.01 mm.

TABLE 3

| Absorbency | Absorbency | Test | Test |
|---|---|---|---|
| Term | Range (g) | Mass (g) | Time (s) |
| Light absorbency | 6 and under | 3.0 | 180 |
| Regular absorbency | 6 to 9 | 5.0 | 300 |
| Super absorbency | 9 to 12 | 8.0 | 480 |
| Super Plus absorbency | 12 to 15 | 11.0 | 660 |
| Ultra absorbency | 15 to 18 | 14.0 | 840 |
| No term | 18 and above | 17.0 | 1020 |

### Fiber Identification

The amount of cotton within the absorbent tampon can be determined via the method described from the American Association of Textile Chemists & Colorists under the name AATCC TM20A-2014.

### Fiber Diameter

Traditionally cotton diameter is expressed in micronaire (actually a gravimetric fineness measurement) and measured as outlined in ASTM D1448-Standard Test Method for Micronaire Reading of Cotton Fibers. The units of micronaire are micrograms per inch and can then be converted to diameter through mathematical manipulation via the equations below.

$$Decitex = 0.393 * Micronaire$$

$$Denier ( microns ) = \sqrt{\frac{( \frac{4 * dtex}{Specific Gravity} )}{π}} * 10$$

where the specific gravity of cotton is 1.54 grams/cubic centimeter.

### Fiber Length

The fiber length of the cotton utilized in the absorbent tampon of the present disclosure may be determined via ANSI/ASTM D1234 utilizing the L (n) methodology which measures the individual fiber length to create an average rather than a weighted average.

### Syngyna

The Syngyna test is carried out as described in 21 CFR § 801.430(f)(2).

### Tampon Efficiency

The Efficiency of the Tampon is calculated as the Syngyna (g) divided by the Dry Tampon Mass (g).

In view of the description above, the following non-limiting combinations are contemplated:

A1. An absorbent tampon having an insertion end and a withdrawal end, the tampon comprising: a primary absorbent, disposed toward the insertion end, wherein the primary absorbent comprises cotton fibers and rayon fibers, and wherein the primary absorbent has an expansion width of greater than 20 mm; a secondary absorbent; and a withdrawal member, wherein said secondary absorbent exhibits a mean length from the primary absorbent of from about 20 mm to about 40 mm, wherein the secondary absorbent exhibits a Mean Segment Mass at a length of 20-30 mm from the primary absorbent of greater than about 9.0 mg, and wherein the mean length from the primary absorbent and mean segment mass are measured according to the Off-Pad Secondary Absorbent Profile method as described herein.

A2. The absorbent tampon according to paragraph A1, wherein the mean segment mass at the length of 20-30 mm from the primary absorbent of the secondary absorbent is from about 9.2 mg to about 20 mg.

A3. The absorbent tampon according to any one of the preceding paragraphs, wherein the secondary absorbent comprises a first segment having a length from the primary absorbent of 0 mm to 10 mm and a second segment having a length from the primary absorbent of 10 mm to 20 mm, and wherein the difference between the mean segment mass of the first segment and the mean segment mass of the second segment is less than 5 mg.

A4. The absorbent tampon according to any one of the preceding paragraphs, wherein the length of 20 mm to 30 mm from the primary absorbent of the secondary absorbent is a third segment, wherein a fourth segment having a length from the primary absorbent of 30 mm to 40 mm, and wherein the difference between the mean segment mass of the third segment and the mean segment mass of the fourth segment is greater than 8 mg.

A5. The absorbent tampon according to any one of the preceding paragraphs, wherein the secondary absorbent exhibits a pusher peak drag force of less than 10 g as measured according to the Tampon Pusher Drag Force method described herein.

A6. The absorbent tampon according to any one of the preceding paragraphs, wherein the secondary absorbent exhibits a pusher peak drag force of less than 85 g as measured according to the Tampon Pusher Drag Force method described herein.

A7. The absorbent tampon according to any one of the preceding paragraphs, wherein the withdrawal member is attached to the primary absorbent and extends beyond the withdrawal end, wherein the mass of secondary absorbent is substantially axially centered about the withdrawal member.

A8. The absorbent tampon according to any one of the preceding paragraphs, wherein the withdrawal member is attached to the primary absorbent and extends beyond the withdrawal end, wherein the secondary absorbent is joined to the withdrawal member.

A9. The absorbent tampon according to any one of the preceding paragraphs, wherein the primary absorbent and the secondary absorbent are configured such that the primary absorbent and the secondary absorbent reside entirely within the vaginal space during use without extending outwardly therefrom.

A10. The absorbent tampon according to any one of the preceding paragraphs, wherein the withdrawal member and the secondary absorbent are stitched to each other.

A11. The absorbent tampon according to any one of the preceding paragraphs, wherein the primary absorbent has a dry tampon mass of less than about 3.0 g.

A12. The absorbent tampon according to any one of the preceding paragraphs, wherein the primary absorbent has a dry tampon mass of less than about 2.6 g.

A13. The absorbent tampon of claim 1, wherein the primary absorbent comprises greater than 50% by weight rayon fibers and less than about 50% by weight cotton fibers.

A14. The absorbent tampon according to any one of the preceding paragraphs, wherein the primary absorbent comprises greater than 80% by weight rayon fibers and less than about 20% by weight cotton fibers.

B1. An absorbent tampon having an insertion end and a withdrawal end, the tampon comprising: a primary absorbent, disposed toward said insertion end, wherein the primary absorbent comprises less than 50% by weight cotton fibers and greater than 50% by weight rayon fibers, and wherein the primary absorbent has an expansion width of greater than 20 mm; a secondary absorbent; and a withdrawal member, wherein said secondary absorbent exhibits a mean length from the primary absorbent of from about 20 mm to about 40 mm, wherein the secondary absorbent exhibits a mean segment mass at a length of 20-30 mm from the primary absorbent of from about 5.0 mg to about 30 mg, wherein the secondary absorbent exhibits a standard deviation of the mean segment mass at the length of 20-30 mm from the primary absorbent of less than about 7 mg, and wherein the mean length from the primary absorbent, mean segment mass, and the standard deviation of the mean segment mass are measured according to the Off-Pad Secondary Absorbent Profile method as described herein.

B2. The absorbent tampon according to paragraph B1, wherein the mean segment mass at the length of 20-30 mm from the primary absorbent of the secondary absorbent is from about 9.0 mg to about 25 mg.

B3. The absorbent tampon according to any one of the preceding claims, wherein the mean segment mass at a length of 30-40 mm from the primary absorbent of the secondary absorbent is from about 1.0 mg to about 5 mg.

B4. The absorbent tampon according to paragraph B1, wherein the primary absorbent comprises less than 20% by weight cotton fibers and greater than 80% by weight rayon fibers.

B5. The absorbent tampon according to paragraph B1, wherein the primary absorbent comprises from about 10% to about 5% by weight cotton fibers and from about 80% to about 95% by weight rayon fibers.

B6. The absorbent tampon according to paragraph B1, wherein the rayon fibers have a round cross sectional shape.

B7. The absorbent tampon according to paragraph B1, wherein the rayon fibers have at least one of a round, trilobal, ellipsoid, stadium, and hollow cross sectional shape.

C1. An absorbent tampon having an insertion end and a withdrawal end, the tampon comprising: a primary absorbent, disposed toward the insertion end, wherein the primary absorbent comprises less than 80% by weight cotton fibers and greater than 20% by weight rayon fibers, and wherein the primary absorbent has an expansion width of greater than 20 mm; a secondary absorbent; and a withdrawal member, wherein said secondary absorbent exhibits a mean length from the primary absorbent of from about 20 mm to about 40 mm, wherein the secondary absorbent comprises a first segment having a length from the primary absorbent of 0 to 10 mm, a second segment having a length from the primary absorbent of from 10 mm to 20 mm, and a third segment having a length from the primary absorbent of from 20 mm to 30 mm, wherein the difference between a mean segment mass of the third segment and the mean segment mass of the fourth segment is at least 8 mg, and wherein the mean length from the primary absorbent and mean segment mass are measured according to the Off-Pad Secondary Absorbent Profile method as described herein.

C2. The absorbent tampon according to paragraph C1, wherein the mean segment mass of the fourth segment is at least about 10% of the mean segment mass of the third segment.

C3. The absorbent tampon according to any one of the preceding claims, comprising an overwrap, wherein the overwrap is disposed on a portion of the primary absorbent.

C4. The absorbent tampon according to any one of the preceding claims, wherein the secondary absorbent is more hydrophilic than the withdrawal member.

C5. The absorbent tampon according to any one of the preceding claims, wherein the second absorbent comprises a material, wherein the material comprises fibers comprising polyester fibers, polyethylene fibers, PET fibers, polypropylene fibers, cotton fibers, rayon fibers, viscose fibers, lyocell fibers, and any combination thereof.

C6. The absorbent tampon according to any one of the preceding claims, wherein the withdrawal member is formed of a section of twisted, braided, or knitted strands or fibers.

C7. The absorbent tampon according to any one of the preceding claims, wherein the mean segment mass of the first segment is less than or equal to the mean segment mass of the second segment.

C8. The absorbent tampon according to any one of the preceding claims, wherein the mean segment mass of the third segment is at least 80% of the mean segment mass of the second segment.

C9. The absorbent tampon according to any one of the preceding claims, comprising a fourth segment having a length from the primary absorbent of from 30 mm to 40 mm, and wherein the mean segment mass of the fourth segment is at least 50% of the mean segment mass of the third segment.

C10. The absorbent tampon according to paragraph C9, wherein the mean segment mass of the fourth segment is greater than 10 mg.

C11. The absorbent tampon according to paragraph C9, wherein the mean segment mass of the fourth segment is less than the mean segment mass of the third segment.

C12. The absorbent tampon according to any one of the preceding claims, wherein the withdrawal member comprises at least two strands.

C13. The absorbent tampon according to paragraph C1, wherein the rayon fibers have a round cross sectional shape.

C14. The absorbent tampon according to paragraph C1, wherein the rayon fibers do not have a trilobal cross sectional shape.

C15. The absorbent tampon according to any one of the preceding claims, wherein the primary absorbent has a compressed tampon length of from about 40-51 mm.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent tampon having an insertion end and a withdrawal end, the tampon comprising:
a primary absorbent, disposed toward the insertion end, wherein the primary absorbent comprises cotton fibers and rayon fibers, and wherein the primary absorbent has an expansion width of greater than 20 mm;
a secondary absorbent; and
a withdrawal member,
wherein said secondary absorbent exhibits a mean length from the primary absorbent of from about 20 mm to about 40 mm,
wherein the secondary absorbent exhibits a Mean Segment Mass at a length of 20-30 mm from the primary absorbent of greater than about 9.0 mg, and
wherein the mean length from the primary absorbent and mean segment mass are measured according to the Off-Pad Secondary Absorbent Profile method as described herein.

2. The absorbent tampon of claim 1, wherein the mean segment mass at the length of 20-30 mm from the primary absorbent of the secondary absorbent is from about 9.2 mg to about 20 mg.

3. The absorbent tampon of claim 1, wherein the secondary absorbent comprises a first segment having a length from the primary absorbent of 0 mm to 10 mm and a second segment having a length from the primary absorbent of 10 mm to 20 mm, and wherein the difference between the mean segment mass of the first segment and the mean segment mass of the second segment is less than 5 mg.

4. The absorbent tampon of claim 1, wherein the length of 20 mm to 30 mm from the primary absorbent of the secondary absorbent is a third segment, wherein a fourth segment having a length from the primary absorbent of 30 mm to 40 mm, and wherein the difference between the mean segment mass of the third segment and the mean segment mass of the fourth segment is greater than 8 mg.

5. The absorbent tampon of claim 1, wherein the secondary absorbent exhibits a pusher peak drag force of less than 10 g as measured according to the Tampon Pusher Drag Force method described herein.

6. The absorbent tampon of claim 1, wherein the secondary absorbent exhibits a pusher peak drag force of less than 85 g as measured according to the Tampon Pusher Drag Force method described herein.

7. The absorbent tampon of claim 1, wherein the withdrawal member is attached to the primary absorbent and extends beyond the withdrawal end, wherein the mass of secondary absorbent is substantially axially centered about the withdrawal member.

8. The absorbent tampon of claim 1, wherein the withdrawal member is attached to the primary absorbent and extends beyond the withdrawal end, wherein the secondary absorbent is joined to the withdrawal member.

9. The absorbent tampon of claim 1, wherein the primary absorbent and the secondary absorbent are configured such that the primary absorbent and the secondary absorbent reside entirely within the vaginal space during use without extending outwardly therefrom.

10. The absorbent tampon of claim 1, wherein the withdrawal member and the secondary absorbent are stitched to each other.

11. The absorbent tampon of claim 1, wherein the primary absorbent has a dry tampon mass of less than about 3.0 g.

12. The absorbent tampon of claim 1, wherein the primary absorbent comprises greater than 50% by weight rayon fibers and less than about 50% by weight cotton fibers.

13. The absorbent tampon of claim 1, wherein the primary absorbent comprises greater than 80% by weight rayon fibers and less than about 20% by weight cotton fibers.

14. An absorbent tampon having an insertion end and a withdrawal end, the tampon comprising:
a primary absorbent, disposed toward said insertion end, wherein the primary absorbent comprises less than 50% by weight cotton fibers and greater than 50% by weight rayon fibers, and wherein the primary absorbent has an expansion width of greater than 20 mm;
a secondary absorbent; and
a withdrawal member,
wherein said secondary absorbent exhibits a mean length from the primary absorbent of from about 20 mm to about 40 mm,
wherein the secondary absorbent exhibits a mean segment mass at a length of 20-30 mm from the primary absorbent of from about 5.0 mg to about 30 mg,
wherein the secondary absorbent exhibits a standard deviation of the mean segment mass at the length of 20-30 mm from the primary absorbent of less than about 7 mg, and
wherein the mean length from the primary absorbent, mean segment mass, and the standard deviation of the mean segment mass are measured according to the Off-Pad Secondary Absorbent Profile method as described herein.

15. The absorbent tampon of claim 14, wherein the mean segment mass at the length of 20-30 mm from the primary absorbent of the secondary absorbent is from about 9.0 mg to about 25 mg.

16. The absorbent tampon of claim 14, wherein the mean segment mass at a length of 30-40 mm from the primary absorbent of the secondary absorbent is from about 1.0 mg to about 5 mg.

17. The absorbent tampon of claim 14, wherein the primary absorbent comprises less than 20% by weight cotton fibers and greater than 80% by weight rayon fibers.

18. The absorbent tampon of claim 14, wherein the primary absorbent comprises from about 10% to about 5% by weight cotton fibers and from about 80% to about 95% by weight rayon fibers.

19. The absorbent tampon of claim 14, wherein the rayon fibers have at least one of a round, trilobal, ellipsoid, stadium, and hollow cross sectional shape.

20. An absorbent tampon having an insertion end and a withdrawal end, the tampon comprising:
a primary absorbent, disposed toward the insertion end, wherein the primary absorbent comprises less than 80% by weight cotton fibers and greater than 20% by weight rayon fibers, and wherein the primary absorbent has an expansion width of greater than 20 mm;
a secondary absorbent; and
a withdrawal member,
wherein said secondary absorbent exhibits a mean length from the primary absorbent of from about 20 mm to about 40 mm,
wherein the secondary absorbent comprises a first segment having a length from the primary absorbent of 0 to 10 mm, a second segment having a length from the primary absorbent of from 10 mm to 20 mm, and a third segment having a length from the primary absorbent of from 20 mm to 30 mm,
wherein the difference between a mean segment mass of the third segment and the mean segment mass of the fourth segment is at least 8 mg, and
wherein the mean length from the primary absorbent and mean segment mass are measured according to the Off-Pad Secondary Absorbent Profile method as described herein.
